(19) United States
(12) Patent Application Publication
(10) Pub. No.: US 2026/0151358 A1
(43) Pub. Date: Jun. 4, 2026

(54) METHODS OF TREATING, AMELIORATING, AND/OR PREVENTING STRESS-RELATED DISORDER

(71) Applicant: Yale University, New Haven, CT (US)

(72) Inventors: Amy F. T. Arnsten, New Haven, CT (US); Dibyadeep Datta, New Haven, CT (US); Mary Kate Joyce, New Haven, CT (US); Min Wang, New Haven, CT (US); Shengtao Yang, New Haven, CT (US)

(21) Appl. No.: 19/127,251

(22) PCT Filed: Nov. 9, 2023
(86) PCT No.: PCT/US2023/079188

§ 371 Date: May 5, 2025

Related U.S. Application Data
(60) Provisional application No. 63/424,811, filed on Nov. 11, 2022.

(51) Int. Cl. A61K 31/138; A61K 31/165; A61K 31/167; A61K 31/17; A61K 31/353; A61K 31/4164; A61P 25/00

(52) U.S. Cl. CPC A61K 31/138; A61K 31/165; A61K 31/167; A61K 31/17; A61K 31/353; A61K 31/4164; A61P 25/00

(57) ABSTRACT

Described herein are methods of treating, ameliorating and/or preventing a stress-related disorder in a subject in need thereof. The method, in certain embodiments, includes administering to the subject a therapeutically effective amount of a selective β-adrenergic receptor antagonist compound. In certain embodiments, the compound has a larger dissociation constant (pKD) for the β1-adrenergic receptor subtype (β1-AR) than for the β2-adrenergic receptor subtype (β2-AR) and/or β3-adrenergic receptor subtype (β3-AR).

### CROSS-REFERENCE TO RELATED APPLICATIONS

The present application claims priority under 35 U.S.C. § 119 (e) to U.S. Provisional Patent Application No. 64/424,811, filed Nov. 11, 2022, which is incorporated herein by reference in its entirety.

### STATEMENT REGARDING FEDERALLY SPONSORED RESEARCH

This invention was made with government support under 2015276 awarded by National Science Foundation. The government has certain rights in the invention.

### BACKGROUND

Exposure to uncontrollable stress impairs higher cognitive functions and top-down control of our attention, behavior and emotion, functions mediated by the recently evolved prefrontal cortex (PFC). Humans exposed to a traumatic stressor often develop post-traumatic stress disorder (PTSD) and/or anxiety disorders, that are debilitating to everyday life, and which involve dysfunction of the PFC.

Stress releases high levels of norepinephrine in the brain, and a common treatment for PTSD is the β-adrenoceptor antagonist, propranolol. However, there is a substantial population of patients who are not helped by propranolol, and propranolol has side effects, such as tiredness and/or aggravation of asthma.

Accordingly, a novel treatment strategy superior to the current propranolol treatment is needed to treat these stress-related disorders. The present disclosure addresses this need.

### SUMMARY

In some aspects, the present disclosure is directed to the following non-limiting embodiments.

The present disclosure provides in one aspect a method of treating, ameliorating, and/or preventing a stress-related disorder in a subject in need thereof. In certain embodiments, the method comprises administering to the subject a therapeutically effective amount of a selective β-adrenergic receptor antagonist compound, wherein the compound has a larger dissociation constant (pK_{D}) for the β1-adrenergic receptor subtype (β1-AR) than for the β2-adrenergic receptor subtype (β2-AR) and/or β3-adrenergic receptor subtype (β3-AR).

The present disclosure provides in one aspect a method of treating, ameliorating, and/or preventing an anxiety disorder in a subject in need thereof. In certain embodiments, the method comprises administering to the subject a therapeutically effective amount of a selective β-adrenergic receptor antagonist compound, wherein the compound has a larger dissociation constant (pK_{D}) for the β1-adrenergic receptor subtype (β1-AR) than for the β2-adrenergic receptor subtype (β2-AR) and/or β3-adrenergic receptor subtype (β3-AR).

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1H: Signaling diagrams and immunoEM, in accordance with some embodiments. FIG. 1A: Schematic illustration of β1-AR/Cav_{1.2} mechanisms mediating the “fight or flight” stress response in the heart, where Cav_{1.2} currents increase internal calcium (Ca²⁺) release through ryanodine receptors (RyR) from the sarcoplasmic reticulum to enhance muscle contraction. FIG. 1B: Schematic illustration of β1-AR/Cav_{1.2} mechanisms in macaque dlPFC mediating stress-induced cognitive impairment. As in heart, β1-AR/Cav_{1.2} are localized on the plasma membrane near the calcium storing SER (the “spine apparatus” in the spine, analogous to the sarcoplasmic reticulum in cardiac muscle), positioned to mediate calcium-induced calcium release. While moderate levels of PKA-calcium signaling are needed to depolarize the synaptic membrane and phosphorylate NMDAR to permit neuronal firing (not shown), high levels of feedforward, calcium-cAMP-PKA-calcium signaling, as occur with β1-AR stimulation during stress, reduce dlPFC neuronal firing by opening SK and HCN-Slack potassium (K⁺) channels. As dlPFC Delay cell firing is needed for working memory, these intracellular signaling events lead to cognitive impairment. FIGS. 1C-1H: ImmunoEM of macaque layer III dlPFC shows expression of Cav_{1.2} (FIGS. 1C-1D), β1-AR (FIGS. 1E-1F) and SK channels (FIGS. 1G-1H) on dendritic spines receiving asymmetric (presumed glutamatergic) synapses, localized on the plasma membrane near the SER spine apparatus (pink pseudocoloring), consistent with the mechanism illustrated in panel b. Sp=spine (pseudocolored yellow): Ax=axon terminal (pseudocolored blue); Mit=mitochondria. Scale bars: 200 nm.

FIGS. 2A-2C: Superficial pyramidal cells co-express Cav_{1.2}, β1-AR and calbindin in macaque and human dlPFC, in accordance with some embodiments. FIG. 2A: MLIF shows co-expression of Cav_{1.2} and β1-AR within the same layer III pyramidal cells in macaque dlPFC: biotin-streptavidin was used to amplify the Cav_{1.2} signal. FIG. 2B: Multiple label immunofluorescence (MLIF) shows co-expression of Cav_{1.2} and calbindin within the same layer III pyramidal cells in macaque dlPFC: biotin-streptavidin was used to amplify the Cav_{1.2} signal. Calbindin expression is much higher in interneurons, but also has moderate expression in a subset of pyramidal cells. FIG. 2C MLIF shows co-expression of β1-AR and calbindin within the same layer III pyramidal cells in macaque dlPFC: biotin-streptavidin was used to amplify the β1-AR signal. Note: the antibodies detecting Cav_{1.2} and SK3 are both created in rabbit: thus, double-labeling could not be used to determine their co-expression within the same pyramidal cells. Scale bars for a-c: large panels 15 μm, small panels 5 μm.

FIGS. 3A-3E: Transcriptomic analyses of human dlPFC reveals subgroups of superficial layer (CUX2-expressing) pyramidal cells with high CACNA1C and an enriched calcium transcriptome. FIG. 3A: A schematic of the methods used to zxc, that co-express high levels of CACNA1C (Cav_{1.2}) with FIG. 3B: ADRB1 (β1-AR): FIG. 3C: CALB1 (calbindin); and FIG. 3D: KCNN3 (SK3 channels). The pyramidal cell subgroups expressing the highest levels of CACNA1C also express GRIN2B (FIG. 3E), which encodes the NMDA receptor with GluN2B subunits that flux high levels of calcium, necessary for dlPFC neurotransmission. Transcriptomic expression levels are all relative to the highest levels of CACNA1C (100%).

FIGS. 4A-4F: Schematic of the methods used to assess drug effects on dlPFC neuronal firing, and the effects of β1-AR on dlPFC Delay cell firing in monkeys performing the ODR working memory task, in accordance with some embodiments. FIG. 4A: zxc, FIG. 4B: zxc, FIG. 4C: zxc, FIG. 4D: zxc, FIG. 4E: The β1-AR agonist, xamoterol (red), reduced delay-related firing of dlPFC Delay cells for the neurons' preferred direction (p=0.003), but not for nonpreferred directions, leading to a significant reduction in the d′ measure of spatial tuning (p=0.0002). (Note, for FIGS. 4E-6C, the left panel shows the firing rate for one, representative neuron's preferred direction, with time in seconds since the onset of the delay period on the x axis, and firing rate of the neuron on the Y axis: control firing is shown in blue. The 0.5 sec cue period is highlighted in dark gray: the delay period begins at “0” sec and is highlighted in light gray. The eye movement response occurs after the delay period. The middle graph shows the average SEM delay-related firing for all Delay cells under control conditions and following drug, and the right graph shows the average±SEM d′ measure of spatial tuning for all Delay cells under control conditions and following drug.) FIG. 4F Conversely, the β1-AR antagonist, betaxolol (green), enhanced delay-related firing (p=0.0009) and increased d′ measures of spatial tuning (p=0.0073).

FIGS. 5A-5C: The effects of LTCC signaling on dlPFC Delay cell firing, and the interaction of LTCC and β1-AR signaling, in accordance with some embodiments. FIG. 5A: Similar to the β1-AR agonist xamoterol, the LTCC channel agonist. (S)-(−)-Bay-K 8644 (S-Bay; red), reduced delay-related firing (p<0.0001) and decreased d′ (p=0.0011). FIG. 5B: The LTCC antagonist, diltiazem (dil), produced an inverted-U dose-response, with low doses of diltiazem (5-20n.A, green) increasing delay-related firing (p=0.0001) and d′ measures of spatial tuning (p=0.0016), while high doses (30-50 nA, red) reduced delay firing for the preferred direction (p<0.0001) and reduced spatial tuning (p=0.0005). FIG. 5C: The average±SEM firing rate of 13 dlPFC Delay cells, showing that the reducing effects of the β1-AR agonist, xamoterol (xamo; p<0.0001), were blocked by pretreatment with the LTCC antagonist, diltiazem (dil: p=0.0015 vs. xamo alone).

FIGS. 6A-6C: The effects of SK or HCN channel blockade on Delay cell firing, in accordance with some embodiments. FIG. 6A: The SK channel blocker, NS8593, enhanced delay-related firing. The firing for a representative neuron's preferred direction is shown (left), with control conditions in blue and NS8593 in green. The middle graph shows the average delay-related firing for 18 Delay cells: NS8593 significantly increased delay firing for the neurons' preferred direction (p<0.0001, as well as a smaller increase for nonpreferred directions, leading to a significant increase in d′ measure of spatial tuning (p=0.0433). FIG. 6B: The average firing rate of 14 dlPFC Delay cells, showing that the reducing effects of the LTCC agonist, S-Bay (p<0.0001), were blocked by pretreatment with the SK channel antagonist, NS8593 (NS: p=0.0104). FIG. 6C: The average firing rate of 12 dlPFC Delay cells, showing that the reducing effects of the LTCC agonist, S-Bay (p<0.0001), were blocked by pretreatment with the HCN channel antagonist, ZD7288 (ZD: p<0.0001).

FIGS. 7A-7B: Pretreatment with systemic administration of a β1-AR antagonist (FIG. 7A) or an LTCC antagonist (FIG. 7B) protects working memory performance from the detrimental effects of stress. Vehicle (Veh) pretreatment in combination with the pharmacological stressor, FG7142 (Str) significantly impaired performance compared to vehicle+vehicle control performance, while pretreatment with the β1-AR antagonist betaxolol (Beta) or the LTCC antagonist, nimodipine (Nim), prevented working memory deficits in FG7142-treated animals. The betaxolol and nimodipine doses were selected to have no effects on their own to preclude additive effects of drug treatment. Results represent mean percent correct on the delayed response task with all individual data points shown (n=6). ***p≤0.001 compared to veh+veh; p<0.001 compared to veh+stress.

FIGS. 8A-8B demonstrate that anti-stress effects of the β1-AR antagonist betaxolol described in FIG. 7A is not limited to betaxolol only, but universal to specific inhibitions of β1-AR, in accordance with some embodiments. FIG. 8A: betaxolol (β1-AR specific antagonist used in FIG. 7A) blocked stress-induced cognitive deficits (working memory deficits) in FG7142-treated animals. FIG. 8B: another β1-AR specific antagonist, nebivolol, also prevented stress-induced cognitive deficits (working memory deficits) in FG7142-treated animals.

FIGS. 9A-9B demonstrate that the β1-AR agonist, xamoterol, reduces PFC neuronal firing (Delay cell firing) in monkeys performing a working memory task (FIG. 9A), while the β2-AR agonist, procaterol, increases delay cell firing (FIG. 9B), in accordance with some embodiments. The data shown in the figures demonstrate that mixed β1-AR and β2-AR blockade is suboptimal for protecting cognition as the two effects cancel each other.

FIGS. 10A-10B demonstrate that the β1-AR antagonist, nebivolol, prevented working memory deficits in FG7142-treated animals, in accordance with some embodiments. 0.1 mg/kg of the β1-AR antagonist, nebivolol, blocked stress-induced cognitive deficits in monkeys (FIG. 10A). 0.01 mg/kg of nebivolol blocked stress-induced cognitive deficits in monkeys (FIG. 10B).

FIG. 11 demonstrates that blocking β1-AR with nebivolol (1.0 mg/kg) improved cognition (working memory), while blocking β2-AR with IC1118551 (0.1 mg/kg) impaired cognition (working memory), in accordance with some embodiments.

FIG. 12 demonstrates that that β1-AR are located on parvalubulmin (PV) expressing GABAergic interneurons in the primate dorsolateral prefrontal cortex, in accordance with some embodiments. In the top panels, multiple label immunofluorescence were used to show that PV interneurons also express β1-AR. The bottom panel is an electron micrograph showing this interaction at the ultrastructural level, where a PV-labeled dendrite also expresses B 1-AR on the plasma membrane, as indicated by the solid arrowhead.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Until now; there has been nothing known about how β-adrenoceptors (β-ARs) alter the functioning of the primate dorsolateral prefrontal cortex (dlPFC) or stress that is linked to this part of brain. Indeed, published data showed that the β antagonist used for treating stress, propranolol, had little effect on dlPFC function (Li et al., Behav Neural Biol. 1994 September: 62 (2): 134-9. doi: 10.1016/s0163-1047 (05) 80034-2).

Unexpectedly, the study described herein (“the present study”) found that the β1-adrenoceptor (β1-AR) is the β-adrenoceptor subtype for causing stress-induced dysfunction of the recently evolved dorsolateral prefrontal cortex (dlPFC) that subserves high order cognitive functions.

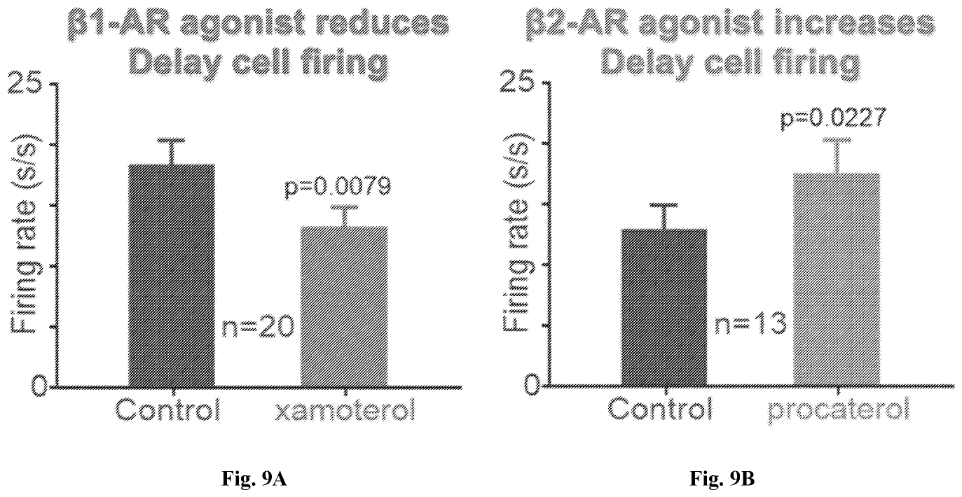

Furthermore, the present study further found that β1-AR stimulation reduces dlPFC neuronal firing during higher cognition, while β2-AR stimulation enhances dlPFC neuronal firing (see e.g., FIGS. 9A-9B). This discovery explains, at least partially, the less than desirable performance of propranolol in treating stress, as well as the data from the study of Li et al. Specifically, propranolol had no effect in dlPFC functions because the inhibitory effects of this compound work on both β1-AR and β2-AR, which canceled out each other. The present study further discovered that compound that selectively block β1-AR are effective in strengthening and protecting dlPFC function and relieving stress and anxiety.

Anxiety disorders interfere with one's ability to function, often leading one to overreact when emotions are triggered. A mix of genetic and environmental factors can raise a person's risk for developing anxiety disorders. Symptoms vary depending on the type of anxiety disorder and include: physical symptoms (cold or sweaty hands, dry mouth, heart palpitations, nausea, numbness or tingling in hands or feet, muscle tension, and/or shortness of breath), mental symptoms (feeling panic, fear and uneasiness, nightmares, repeated thoughts or flashbacks of traumatic experiences, and/or uncontrollable and obsessive thoughts), and behavioral symptoms (inability to be still and calm, ritualistic behaviors, such as washing hands repeatedly, and/or trouble sleeping).

In certain embodiments, experiencing much stress over a long period of time can lead to an anxiety disorder, but anxiety disorders are considered to be distinct from stress disorders. In certain embodiments, the anxiety disorder comprises generalized anxiety disorder (GAD) (fear of the future, anxiety regarding altered schedules, a sense of incompleteness, and so forth; this disorder might have physical implications such as fatigue, sleeping disorders, increased heart rate, inability to focus, muscle stress or soreness, and/or worry). In certain embodiments, the anxiety disorder comprises panic disorder (a recurring experience of extreme stress, anxiety, and worry: most commonly connected with erratic anxiety attacks or other intense sensations of stress, anxiety, or fear). In certain embodiments, the anxiety disorder comprises a phobia (an intense fear of an item, person, place, or thing; different phobias may consist of the fear of flying, clowns, spiders, public speaking, enclosures, and more). In certain embodiments, the anxiety disorder comprises agoraphobia (feeling of intense fear where escape may be challenging or uncomfortable to experience; may also include the fear of being unaccompanied outdoors and/or fear of leaving home or being alone). In certain embodiments, the anxiety disorder comprises separation anxiety disorder (an extreme, illogical worry of being separated from a loved one; this worry can arise when the person is present or absent, e.g., fear of them leaving for a specific reason, or fear of them being harmed while apart). In certain embodiments, the anxiety disorder comprises social anxiety disorder (encompasses public speaking, fear of remaining in public spaces, aversions to social interactions, fear of eating or drinking in public, fear of casual social settings, and/or fear of leaving the house).

In certain embodiments, the stress-related disorder comprises obsessive-compulsive disorder (OCD). In certain embodiments, the stress-related disorder comprises post-traumatic stress-disorder (the result of past traumatic events significantly impacting the person's mental health; this is often the result of reliving, remembering, or having nightmares associated with past trauma). In certain embodiments, the stress-related disorder comprises reactive attachment disorder (RAD) (an inability to express emotional or physical attachment to others: in children, symptoms may include a disinterest in physical or emotional comfort when distressed or a lack of responsive emotions—at times, this is a result of neglect and lack of proper caregiving in order for the child to develop relationships: in adults, these symptoms may increase and hinder them from forming close relationships and potentially lead to other mental illnesses). In certain embodiments, the stress-related disorder comprises disinhibited social engagement disorder (a stress-related disorder characterized by behavior deemed culturally and socially inappropriate; this can include inappropriate behavior, oversharing intimate information, or close, physical familiarity with strangers). In certain embodiments, the stress-related disorder comprises acute stress disorder (similar in effect to PTSD, but typically holds a shorter duration following the stressful situation). In certain embodiments, the stress-related disorder comprises adjustment disorder (a disorder that presents itself with symptoms with an identifiable cause: these may include work-related stressors, moving to a different state, environmental or lifestyle changes, or educational shifts—adjustment disorder is typically a short-term experience and changes as the person becomes more comfortable with the change).

Accordingly, in some embodiments, the present disclosure is directed to a method of treating, ameliorating and/or preventing a stress-related disorder. In some embodiments, the present disclosure is directed to a method of treating, ameliorating and/or preventing an anxiety disorder.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting: information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or +10%, in certain embodiments±5%, in certain embodiments±1%, in certain embodiments±0.1% from the specified value, as such variations are appropriate to perform the disclosed methods.

As used herein, the term “Bay K8644” refers to methyl 2,6-dimethyl-5-nitro-4-[2-(trifluoromethyl)phenyl]-1,4-dihydropyridine-3-carboxylate, or a salt, stereoisomer, or solvate thereof:

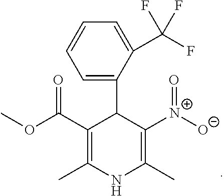 [SMILES: COC(=O)C1=C(C)NC(C)=C([N+](=O)[O-])C1c1ccccc1C(F)(F)F]

As used herein, the term “(S)-(−)-Bay-K 8644” refers to (−)-methyl 2,6-dimethyl-5-nitro-4 (S)-[2-(trifluoromethyl)phenyl]-1,4-dihydropyridine-3-carboxylate, or a salt, stereoisomer, or solvate thereof:

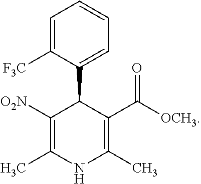 [SMILES: COC(=O)C1=C(C)NC(C)=C([N+](=O)[O-])[C@H]1c1ccccc1C(F)(F)F]

As used herein, the term “diltiazem” refers to cis-(+)-[2-(2-dimethylaminoethyl)-5-(4-methoxyphenyl)-3-oxo-6-thia-2-azabicyclo[5.4.0]undeca-7,9,11-trien-4-yl]ethanoate, or a salt, stereoisomer, or solvate thereof:

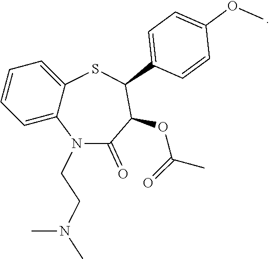 [SMILES: COc1ccc([C@@H]2Sc3ccccc3N(CCN(C)C)C(=O)[C@@H]2OC(C)=O)cc1]

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “FG7142,” “FG-7142,” “ZK31906,” or “ZK-31906” refers to N-methyl-5H-pyrido[3,4-b]indole-2-carboxamide, or a salt, stereoisomer, or solvate thereof:

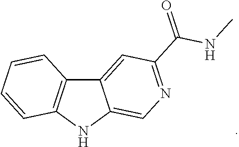 [SMILES: CNC(=O)c1cc2c(cn1)[nH]c1ccccc12]

As used herein, the term “IC1118551” refers to 3-(isopropylamino)-1-[(7-methyl-4-indanyl)oxy]butan-2-ol, or a salt, stereoisomer, or solvate thereof:

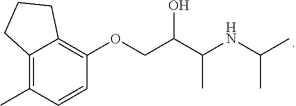 [SMILES: Cc1ccc(OCC(O)C(C)NC(C)C)c2c1CCC2]

As used herein, the term “nimodipine” refers to 3-(2-methoxyethyl) 5-propan-2-yl 2,6-dimethyl-4-(3-nitrophenyl)-1,4-dihydropyridine-3,5-dicarboxylate, or a salt, stereoisomer, or solvate thereof:

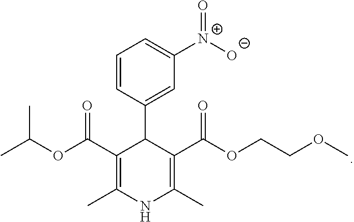 [SMILES: COCCOC(=O)C1=C(C)NC(C)=C(C(=O)OC(C)C)C1c1cccc([N+](=O)[O-])c1]

As used herein, the term “NS8593” refers to N-[(1R)-1,2,3,4-tetrahydro-1-naphthalenyl]-1H-benzimidazol-2-amine, or a salt, stereoisomer, or solvate thereof:

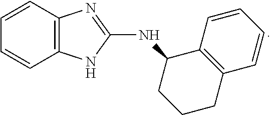 [SMILES: c1ccc2c(c1)CCC[C@H]2Nc1nc2ccccc2[nH]1]

As used herein, the term “patient,” “individual,” or “subject” refers to a human or a non-human mammal. Non-human mammals include, for example, livestock and pets, such as ovine, bovine, porcine, canine, feline and murine mammals. In certain embodiments, the patient, individual or subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt or solvate of the compound useful within the disclosure. Other additional ingredients that may be included in the pharmaceutical compositions used in the practice of the disclosure are known in the art and described, for example in Remington's Pharmaceutical Sciences (Genaro. Ed., Mack Publishing Co., 1985. Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid, Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic. β-hydroxybutyric, salicylic, galactaric and galacturonic acid. Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “procaterol” refers to (±)-(1R,2S)-rel-8-hydroxy-5-[1-hydroxy-2-(isopropylamino)butyl]-quinolin-2 (1H)-one, or a salt, stereoisomer, or solvate thereof:

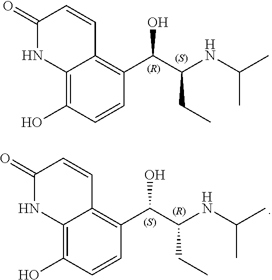

As used herein, the term “propranolol” refers to (RS)-1-(1-methylethylamino)-3-(1-naphthyloxy)propan-2-ol, or a salt, stereoisomer, or solvate thereof:

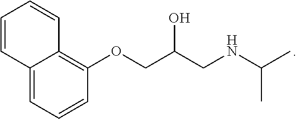 [SMILES: CC(C)NCC(O)COc1cccc2ccccc12]

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder, and/or a symptom of a disease or disorder, with the purpose to cure, heal, alleviate, relieve, alter, remedy, ameliorate, improve, or affect the disease or disorder, and/or the symptoms of the disease or disorder. Such treatments may be specifically tailored or modified, based on knowledge obtained from the field of pharmacogenomics.

As used herein, the term “xamoterol” refers to (RS)—N-(2-{[2-hydroxy-3-(4-hydroxyphenoxy)propyl]amino}ethyl)morpholine-4-carboxamide, or a salt, stereoisomer, or solvate thereof:

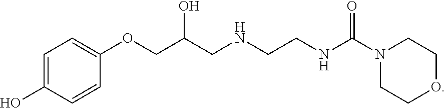 [SMILES: O=C(NCCNCC(O)COc1ccc(O)cc1)N1CCOCC1]

As used herein, the term “ZD7288” refers to N-ethyl-1,6-dihydro-1,2-dimethyl-6-(methylimino)-N-phenyl-4-pyrimidinamine, or a salt, stereoisomer, or solvate thereof:

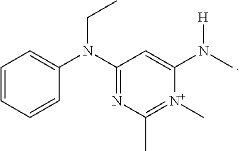 [SMILES: CCN(c1ccccc1)c1cc(NC)[n+](C)c(C)n1]

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Method of Treating, Ameliorating and/or Preventing Stress-Related Disorder and/or Anxiety Disorder

In some embodiments, the instant specification is directed to a method of treating, ameliorating, and/or preventing a stress-related disorder in a subject in need thereof. In some embodiments, the method includes administering to the subject a therapeutically effective amount of a selective β1-adrenergic receptor antagonist.

In some embodiments, the instant specification is directed to a method of treating, ameliorating, and/or preventing an anxiety disorder in a subject in need thereof. In some embodiments, the method includes administering to the subject a therapeutically effective amount of a selective β1-adrenergic receptor antagonist.

In some embodiments, the dissociation constant (pK_{D}) of the selective β1-adrenergic receptor antagonist for β1-adrenergic receptor subtype (β1-AR) is larger than the pK_{D} of the selective β1-adrenergic receptor antagonist for β2-adrenergic receptor subtype (β2-AR) and/or the pK_{D} of the selective β1-adrenergic receptor antagonist for β3-adrenergic receptor subtype (β3-AR).

In some embodiments, the dissociation constant (pK_{D}) of the selective β1-adrenergic receptor antagonist for β1-adrenergic receptor subtype (β1-AR) is larger than the pK_{D} of the selective β1-adrenergic receptor antagonist for β2-adrenergic receptor subtype (β2-AR).

In some embodiments, the compound contemplated in the disclosure has an inhibition constant (pK_{D}) for the β1-adrenergic receptor subtype (β1-AR) that is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0 units or higher than for the β2-adrenergic receptor subtype (β2-AR).

In some embodiments, the compound contemplated in the disclosure has an inhibition constant (pK_{D}) for the β1-adrenergic receptor subtype (β1-AR) that is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0 units or higher than for the β3-adrenergic receptor subtype (β3-AR).

In some embodiments, the compound contemplated in the disclosure has an dissociation constant (pK_{D}) for the β1-adrenergic receptor subtype (β1-AR) about 6.2 units or higher, such as about 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9 or 11.0 units or higher.

In some embodiments, the compound contemplated in the disclosure is selective toward β1-adrenergic receptor subtype (β1-AR) over the β2-adrenergic receptor subtype (β2-AR) and/or the β3-adrenergic receptor subtype (β3-AR).

In some embodiments, the compound contemplated in the disclosure is about two times or more selective for the β1-adrenergic receptor subtype (β1-AR) than for the β2-adrenergic receptor subtype (β2-AR), such as about 3 times or more, 5 times or more, 10 times or more, 20 times or more. 50 times or more, 75 times or more, 100 times or more, 200 times or more, 500 times or more, or 1000 times or more selective for the β1-adrenergic receptor subtype (β1-AR) than for the β2-adrenergic receptor subtype (β2-AR).

In some embodiments, the compound contemplated in the disclosure is about two times or more selective for the β1-adrenergic receptor subtype (β1-AR) than for the β3-adrenergic receptor subtype (β3-AR), such as about 3 times or more, 5 times or more, 10 times or more, 20 times or more, 50 times or more, 75 times or more. 100 times or more. 200 times or more, 500 times or more selective for the β1-adrenergic receptor subtype (β1-AR) than for the β3-adrenergic receptor subtype (β3-AR).

In some embodiments, selective β1-adrenergic receptor subtype antagonist compound comprises at least one selected from the group consisting of acebutolol, atenolol, betaxolol, bisoprolol, CGP 20712A, esmolol. ICI 89406, metoprolol, nebivolol, and practolol, or a salt, stereoisomer, or solvate thereof.

In some embodiments, the selective β1-adrenergic receptor antagonist includes acebutolol (also known as N-{3-acetyl-4-[2-hydroxy-3-(propan-2-ylamino)propoxy]phenyl}butanamide). Acebutolol has a pK_{D} value of 6.46±0.03 for β1-AR, a pK_{D} value of 6.08±0.07 for β2-AR, and a pK_{D} value of 4.41±0.12 for β3-AR. Acebutolol has about 2.4 times selectivity for β1-AR over β2-AR, and about 112.2 times selectivity for β1-AR over β3-AR (Baker et al., British Journal of Pharmacology (2005) 144, 317-322):

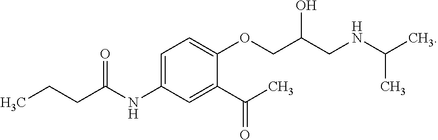 [SMILES: CCCC(=O)Nc1ccc(OCC(O)CNC(C)C)c(C(C)=O)c1]

In some embodiments, the selective β1-adrenergic receptor antagonist includes atenolol (also known as 2-(4-{2-hydroxy-3-[(propan-2-yl)amino]propoxy}phenyl)acetamide). Atenolol has a pK_{D} value of 6.66±0.05 for β1-AR, a pK_{D} value of 5.99±0.14 for β2-AR, and a pK_{D} value of 4.11±0.07 for β3-AR. Atenolol has about 4.7 times selectivity for β1-AR over β2-AR, and about 354.8 times selectivity for β1-AR over β3-AR (Baker et al., British Journal of Pharmacology (2005) 144, 317-322):

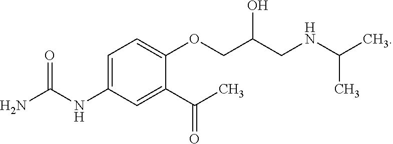 [SMILES: CC(=O)c1cc(NC(N)=O)ccc1OCC(O)CNC(C)C]

In some embodiments, the selective β1-adrenergic receptor antagonist includes betaxolol (also known as 1-{4-[2-(cyclopropylmethoxy)ethyl]phenoxy}-3-(propan-2-ylamino)propan-2-ol). Betaxolol has a pK_{D} value of 8.21±0.07 for β1-AR, a pK_{D} value of 7.38±0.06 for β2-AR, and a pK_{D} value of 5.97±0.08 for β3-AR. Betaxolol has about 6.8 times selectivity for β1-AR over β2-AR, and about 173.8 times selectivity for β1-AR over β3-AR (Baker et al., British Journal of Pharmacology (2005) 144, 317-322):

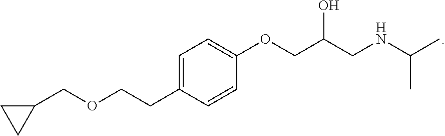 [SMILES: CC(C)NCC(O)COc1ccc(CCOCC2CC2)cc1]

In some embodiments, the selective β1-adrenergic receptor antagonist includes bisoprolol (also known as 1-{4-[(2-isopropoxyethoxy)methyl]phenoxy}-3-(isopropylamino)propan-2-ol). Bisoprolol has a pK_{D} value of 7.83±0.04 for β1-AR, a pK_{D} value of 6.70±0.05 for β2-AR, and a pK_{D} value of 5.67±0.10 for β3-AR. Bisoprolol has about 13.5 times selectivity for β1-AR over β2-AR, and about 144.5 times selectivity for β1-AR over β3-AR (Baker et al., British Journal of Pharmacology (2005) 144, 317-322):

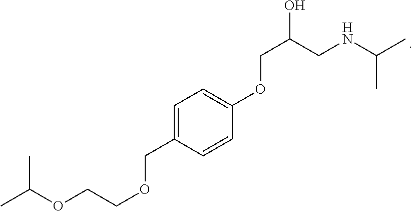 [SMILES: CC(C)NCC(O)COc1ccc(COCCOC(C)C)cc1]

In some embodiments, the selective β1-adrenergic receptor antagonist includes CGP 20712A (also known as 1-[2-((3-carbamoyl-4-hydroxy)phenoxy)ethylamino]-3-[4-(1-methyl-4-trifluoromethyl-2-imidazolyl)phenoxy]-2-propanol). CGP 20712A has a pK_{D} value of 8.81±0.03 for β1-AR, a pK_{D} value of 6.11±0.05 for β2-AR, and a pK_{D} value of 5.19±0.09 for β3-AR. CGP 20712A has about 501.2 times selectivity for β1-AR over β2-AR, and about 4168.7 times selectivity for β1-AR over β3-AR (Baker et al., British Journal of Pharmacology (2005) 144, 317-322):

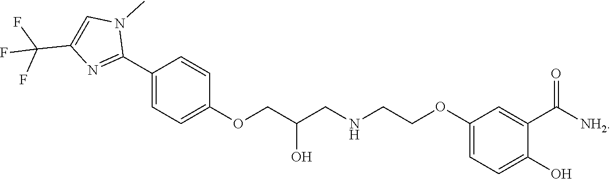 [SMILES: Cn1cc(C(F)(F)F)nc1-c1ccc(OCC(O)CNCCOc2ccc(O)c(C(N)=O)c2)cc1]

In some embodiments, the selective β1-adrenergic receptor antagonist includes esmolol (also known as methyl 3-{4-[2-hydroxy-3-(propan-2-ylamino)propoxy]phenyl}propanoate) (Cojocariu et al., Medicina (Kaunas) (2021) 57 (2), 155:

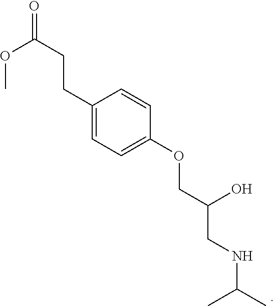 [SMILES: COC(=O)CCc1ccc(OCC(O)CNC(C)C)cc1]

In some embodiments, the selective β1-adrenergic receptor antagonist includes ICI 89406 (also known as 1-[2-[[3-(2-cyanophenoxy)-2-hydroxypropyl]amino]ethyl]-3-phenylurea). ICI 89406 has a pK_{D} value of 8.91±0.09 for β1-AR, a pK_{D} value of 7.07±0.06 for β2-AR, and a pK_{D} value of 5.69±0.06 for β3-AR. ICI 89406 has about 69.2 times selectivity for β1-AR over β2-AR, and about 1659.6 times selectivity for β1-AR over β3-AR (Baker et al., British Journal of Pharmacology (2005) 144, 317-322):

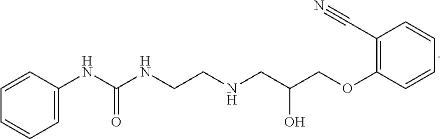 [SMILES: N#Cc1ccccc1OCC(O)CNCCNC(=O)Nc1ccccc1]

In some embodiments, the selective β1-adrenergic receptor antagonist includes metoprolol (also known as 1-[4-(2-methoxyethyl)phenoxy]-3-(propan-2-ylamino)propan-2-ol). Metoprolol has a pK_{D} value of 7.26=0.07 for β1-AR, a pK_{D} value of 6.89±0.09 for β2-AR, and a pK_{D} value of 5.16±0.12 for β3-AR. Metoprolol has about 2.3 times selectivity for β1-AR over β2-AR, and about 125.9 times selectivity for β1-AR over β3-AR (Baker et al., British Journal of Pharmacology (2005) 144, 317-322):

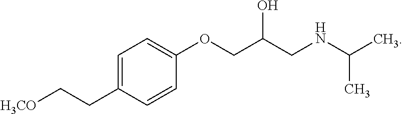 [SMILES: COCCc1ccc(OCC(O)CNC(C)C)cc1]

In some embodiments, the selective β1-adrenergic receptor antagonist includes nebivolol (also known as 1-(6-fluoro-3,4-dihydro-2H-chromen-2-yl)-2-[[2-(6-fluoro-3,4-dihydro-2H-chromen-2-yl)-2-hydroxyethyl]amino]ethanol), which is approximately 3.5 times more β1-AR-selective than bisoprolol in the human myocardium (and thus at least about 3.5×13.5=about 47.3 times selectivity for β1-AR over β2-AR) (Bundkirchen et al., European J. Pharmacology (2003) 460 (1), 19-26):

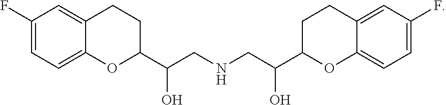 [SMILES: OC(CNCC(O)C1CCc2cc(F)ccc2O1)C1CCc2cc(F)ccc2O1]

In some embodiments, the selective β1-adrenergic receptor antagonist includes practolol (also known as N-{4-[2-hydroxy-3-(propan-2-ylamino)propoxy]phenyl}acetamide). Practolol has a pK_{D} value of 6.14±0.05 for β1-AR, a pK_{D} value of 4.99±0.07 for β2-AR, and a pK_{D} value of >4 for β3-AR. Practolol has about 14.1 times selectivity for β1-AR over β2-AR, and about 588.8 times selectivity for β1-AR over β3-AR (Baker et al., British Journal of Pharmacology (2005) 144, 317-322):

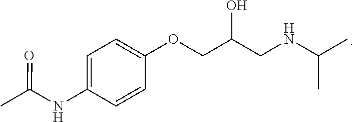 [SMILES: CC(=O)Nc1ccc(OCC(O)CNC(C)C)cc1]

The compounds contemplated in the disclosure may possess one or more stereocenters, and each stereocenter may exist independently in either the (R) or(S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds contemplated in the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

In some embodiments, the disclosure contemplates the use of any compound described herein, or a salt, solvate, enantiomer, enantiomeric mixture, diastereoisomer, diastereoisomeric mixture, geometric isomer, tautomer, and/or isotopically labelled isomer thereof, and/or any mixture thereof.

In some embodiments, the stress-related disorder is a stress-induced prefrontal cortical dysfunction.

In some embodiments, the stress-related disorder comprises post-traumatic stress disorder (PTSD). In some embodiments, the stress-related disorder comprises an agitation/disruptive/aggressive behavior, including disruptive behaviors associated with dementia.

In some embodiments, the subject is a mammal, such as a primate, such as a human.

### Combination Therapies

The compounds of the present disclosure are intended to be useful in the methods of present disclosure in combination with one or more additional compounds useful for treating, ameliorating, and/or preventing any of the diseases or disorders contemplated within the disclosure. These additional compounds may comprise compounds of the present disclosure or compounds, e.g., commercially available compounds, known to treat, prevent, or reduce the symptoms of the diseases or disorders contemplated within the disclosure.

A synergistic effect may be calculated, for example, using suitable methods such as, for example, the Sigmoid-Eₘₐₓ equation (Holford & Scheiner, 19981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions useful within the present disclosure to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient: the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions useful within the disclosure are formulated using one or more pharmaceutically acceptable excipients or carriers. In certain embodiments, the pharmaceutical compositions of the disclosure comprise a therapeutically effective amount of a compound useful within the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions useful within the disclosure are administered to the patient in dosages that range from one to five times per day or more. In other embodiments, the compositions useful within the disclosure are administered to the patient in range of dosages that include, but are not limited to, once every day, every two, days, every three days to once a week, and once every two weeks. It will be readily apparent to one skilled in the art that the frequency of administration of the various combination compositions useful within the disclosure will vary from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

Compounds for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In certain embodiments, the dose of a compound is from about 1 mg and about 2,500 mg. In other embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in certain embodiments, a dose of a second compound (i.e., a drug used for treating a disease or disorder) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other cognition improving agents.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

Non-limiting examples of formulations useful within the disclosure, including formulations of prodrugs of compounds useful within the disclosure, are recited in the following patent application publications, each of which is incorporated by reference in its entirety herein: US 2012/0065152; WO 2011/033296; US 2011/0065796; and WO 2007/016284.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose: granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon. West Point. Pa. (e.g., OPADRY™ OY Type, OYC Type. Organic Enteric OY-P Type. Aqueous Enteric OY-A Type. OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds of the disclosure, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Parenteral Administration

For parenteral administration, the compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Application Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In a preferred embodiment of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound will depend on the age, sex and weight of the patient, the current medical condition of the patient and the progression of cognitive changes in the patient being treated. The skilled artisan will be able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: L-Type Calcium Channel CACNA1C Impairs Dorsolateral Prefrontal Cognition

Genetic variations in L-type-calcium-channel (LTCC) Cav_{1.2} (CACNA1C) are associated with cognitive impairment and risk of mental disorders, but the cellular basis is unknown. The current study examined Cav_{1.2}-LTCC actions in macaque dorsolateral prefrontal cortical (dlPFC) NMDAR-GluN2B circuits that mediate cognition, and found actions similar to heart: LTCCs were essential for working memory-related neuronal firing, but high levels, driven by β1-adrenoceptor (β1-AR) stress-like signaling, reduced firing via SK channel opening, with stress-induced cognitive deficits blocked by β1-AR or LTCC antagonists. A calcium-enriched transcriptome was also identified in human dlPFC pyramidal cells with high CACNA1C, co-expressing GRIN2B (NMDAR-GluN2B). KCNN3 (SK3 channels), ADRB1 (β1-AR), and uniquely co-expressing CALB1 (calbindin), a pattern recapitulated with protein expression in macaque layer III spines. As calbindin-expressing dlPFC pyramidal cells are selectively vulnerable to Alzheimer's pathology when calbindin is lost with age, this subgroup of neurons with amplified Cav_{1.2} signaling may be especially susceptible to toxic calcium actions.

### Example 2

Genetic variations in the L-type calcium channel (LTCC) Cav_{1.2}, which is encoded by CACNA1C, are consistently associated with cognitive deficits and increased risk of mental disorders, but the reason why this channel is so important to higher cognition is not known. CACNA1C alterations have been linked to increased risk of schizophrenia, bipolar disorder, PTSD, and Alzheimer's disease (AD), all of which are characterized by cognitive deficits and are exacerbated by stress exposure. Genetic studies suggest that Cav_{1.2} may play an especially important role in the functioning of the dorsolateral PFC (dlPFC), a recently evolved brain region in primates that subserves working memory and higher cognitive operations. For example, the rs1006737 variant in CACNA1C has been shown to increase Cav_{1.2} mRNA, especially in the dlPFC, and is associated with inefficient activation of the dlPFC during working memory that is normalized by LTCC blockade. This variant is also associated with increased risk of schizophrenia, a disorder with prominent working memory deficits and dlPFC dendritic spine loss focused in layer III. Layer III dlPFC is also the focus of tau pathology in AD, with calbindin-expressing pyramidal cells especially vulnerable to degeneration when calbindin is lost with age. However, it is not known why this subset of neurons would be so vulnerable to atrophy, or why alterations in Cav_{1.2} expression and/or function would particularly impair dlPFC cognition. These questions cannot be properly addressed in mouse models, as rodents do not have a dlPFC.

The higher cognitive operations of the dlPFC arise from its ability to represent information without sensory stimulation, e.g., as has been shown with dlPFC “Delay cells” which represent a location in space during a visuospatial working memory task. Delay cells can sustain spatially-tuned, persistent firing across the delay period for multiple seconds through extensive, recurrent excitatory pyramidal cell circuits in layer III of dlPFC, which excite each other via NMDA receptors (NMDARs) on dendritic spines, including large reliance on NMDAR with GluN2B subunits (GRIN2B) that flux the highest levels of calcium, but little AMPAR contribution. Elevated cytosolic calcium levels may be needed to depolarize the postsynaptic membrane to support prolonged, NMDAR-mediated neuronal firing needed for working memory. However, the role of Cav_{1.2} LTCCs in these recently evolved circuits is not known.

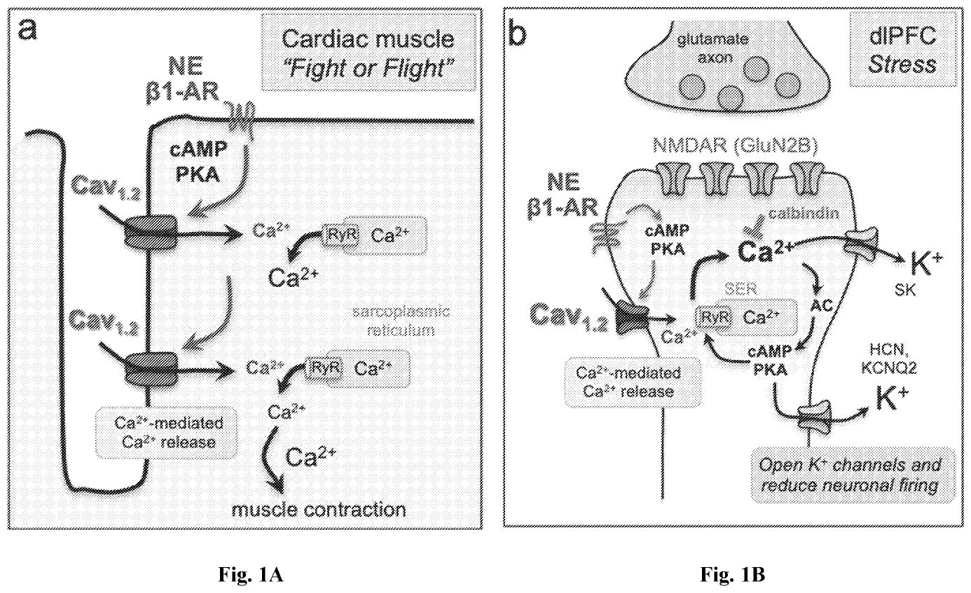

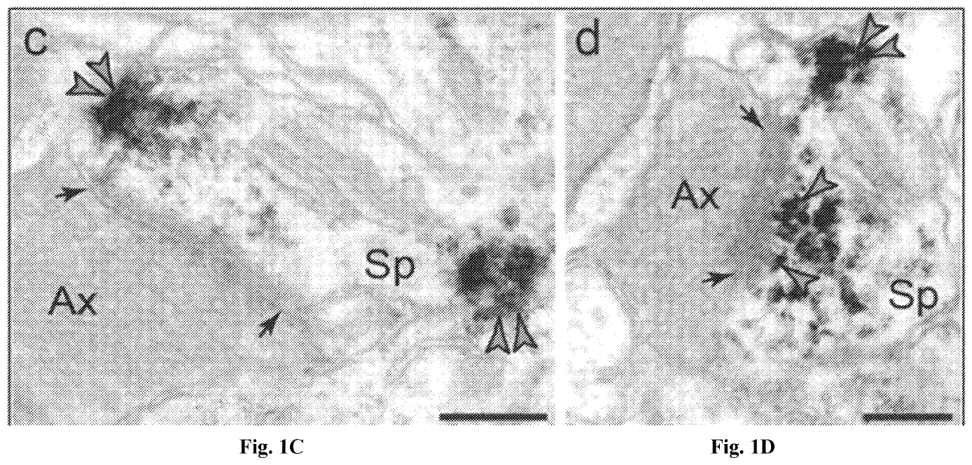

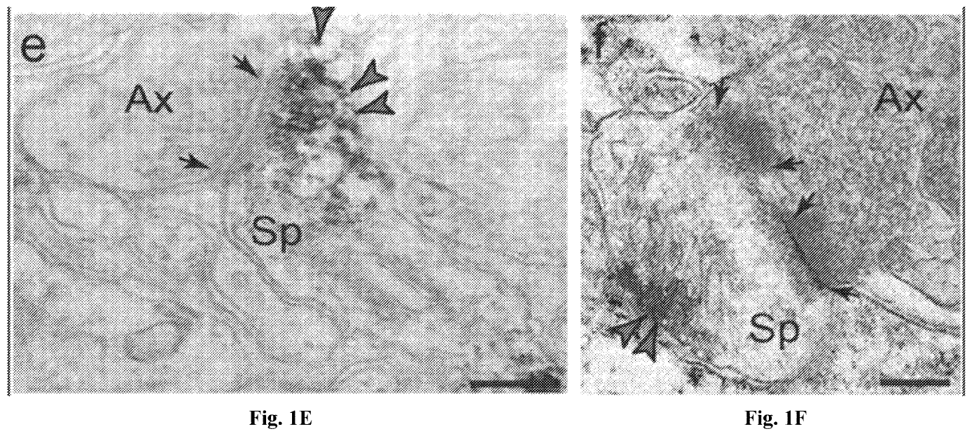

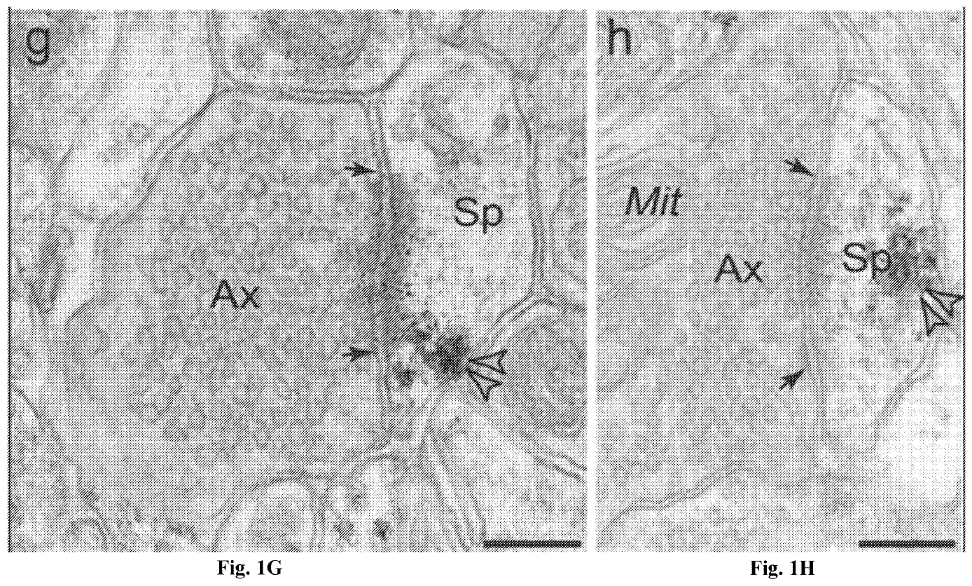

Important clues may come from studies of cardiac muscle, where Cav_{1.2} LTCCs are needed for normal cardiac excitation-contraction coupling and action potential duration, but which also play a key role in the “fight or flight” stress response in heart (FIG. 1A). This work has shown that high levels of norepinephrine (NE) and epinephrine release during stress stimulate noradrenergic β1-adrenoceptors (β1-ARs; ADRB1), which activate Cav_{1.2} currents via PKA signaling. Cav₁₂ currents in turn drive calcium-mediated calcium release from the sarcoplasmic reticulum through ryanodine receptors (RyR) to enhance muscle contraction. As β1-ARs have low affinity for NE, they are only activated under conditions of higher NE release, e.g. during psychological or physiological stress exposure, when NE release is increased from the sympathetic nervous system and in PFC. The dlPFC may share some of these properties with cardiac muscle (FIG. 1B), as dlPFC layer III dendritic spines express the molecular machinery for cAMP magnification of internal calcium release from the smooth endoplasmic reticulum (SER, the spine apparatus in the spine), which is analogous to the sarcoplasmic reticulum in the heart. During stress, high levels of cAMP-PKA signaling reduce dlPFC neuronal firing by opening HCN/Slack and/or KCNQ2 channels, respectively. However, the roles of voltage-gated calcium channels and β1-AR signaling in dlPFC have never been explored, even though the human genetic data suggest that Cav_{1.2} may play an especially important role in primate dlPFC. Thus, the current study examined the localization and physiological effects of Cav_{1.2} LTCCs and β1-ARs in the macaque dlPFC, and related these findings to transcriptomic analyses of pyramidal cells in the human dlPFC

### Example 3: Localization of Cav_{1.2} in Rhesus dlPFC

The first set of experiments determined whether Cav_{1.2} and related signaling proteins were localized on macaque layer III dlPFC spines, which are thought to be key to the recurrent excitation needed for persistent firing during working memory. Immunoelectron microscopy (immunoEM) of perfusion-fixed macaque dlPFC was employed to localize Cav_{1.2} and related signaling proteins at the ultrastructural level. These experiments revealed that both Cav_{1.2} (FIGS. 1C-1D) and β1-AR (FIGS. 1E-IF) are concentrated on layer III dlPFC spines, on plasma membranes near the SER spine apparatus, positioned to drive internal calcium release. This spatial relationship is similar to that seen with Cav_{1.2} and the sarcoplasmic reticulum in the heart. In addition to dendritic spines, β1-AR and Cav_{1.2} were also found on the plasma membrane of pyramidal cell dendrites, but rarely on axons. They were also seen in glial leaflets (likely astrocytes). SK3 channels, whose open state is increased by calcium, were also seen on layer III dlPFC spine membranes (FIGS. 1G-1H), as well as on pyramidal-like dendritic shafts. Layer III dlPFC spine membranes also express HCN1 and KCNQ2 channels, whose open state is increased by cAMP and PKA signaling, respectively, reducing Delay cell. Thus, layer III dlPFC spines express a constellation of β1-AR and Cav_{1.2} on spine membranes positioned to increase calcium-cAMP opening of nearby K⁺ channels.

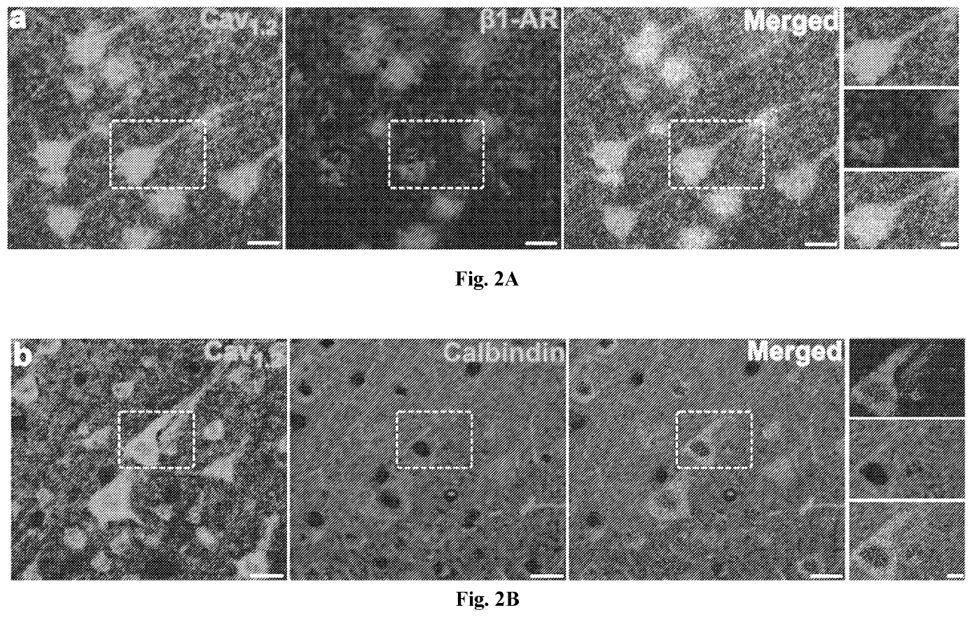

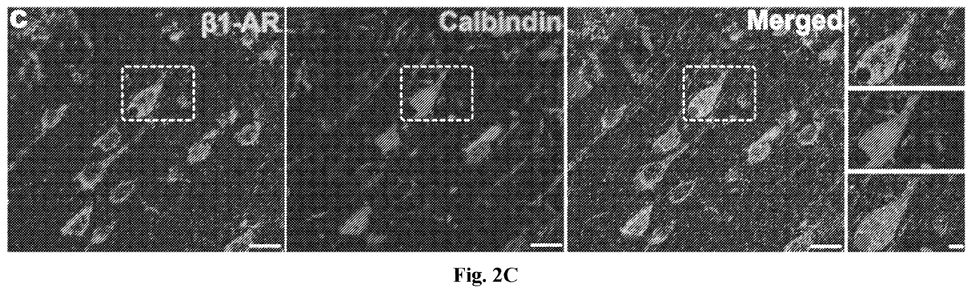

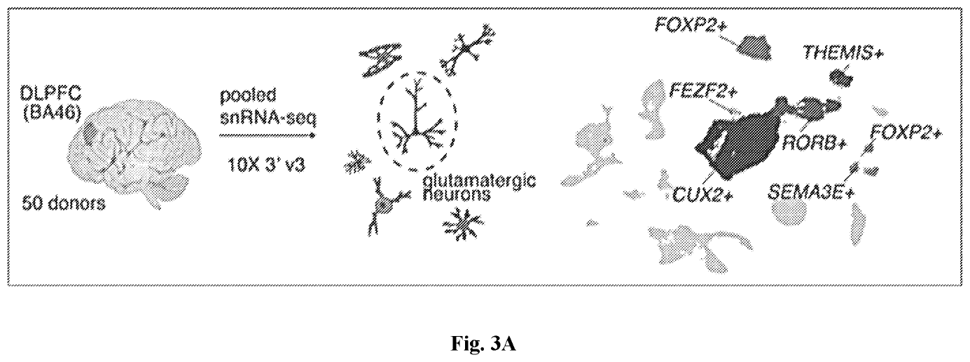

Co-localization of Cav_{1.2} with related proteins within the same pyramidal cell was examined using multiple label immunofluorescence. These data showed that the same rhesus layer III dlPFC pyramidal cells that express Cav_{1.2} also express β1-AR (FIG. 2A), and the calcium-binding protein, calbindin (FIG. 2B), which may be needed to regulate high levels of calcium in the cytosol. FIG. 2C shows that the calbindin-expressing pyramidal cells also express β1-AR. (SK3 channel co-localization could not be examined due to incompatibility of available antibodies for dual labeling.) These pyramidal cells are focused in layer III, consistent with reports from human dlPFC. Thus, the data indicate a subpopulation of pyramidal cells in macaque layer III dlPFC that co-express Cav_{1.2}, β1-AR and calbindin.

### Example 4: Similar Pyramidal Cells in Human dlPFC

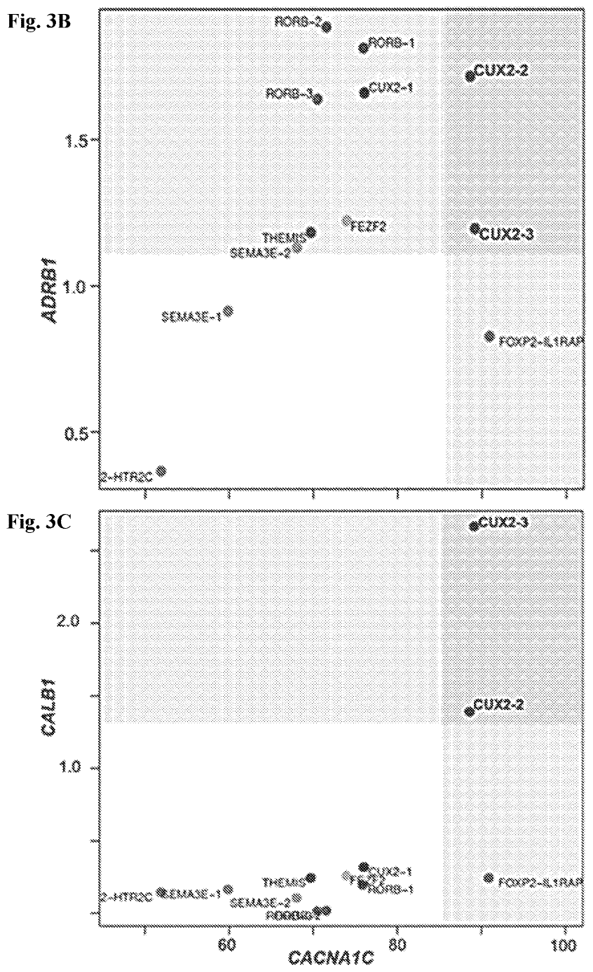

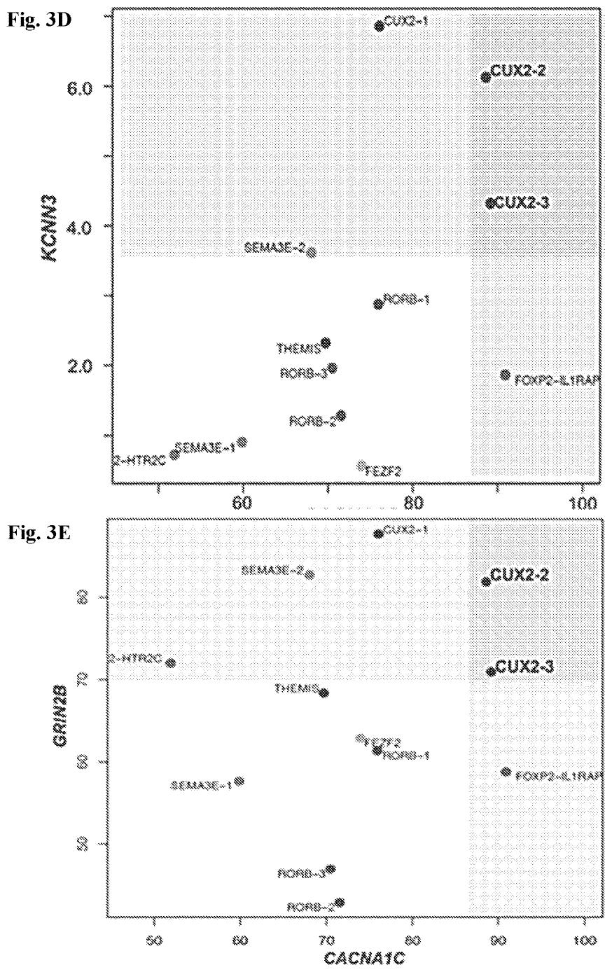

A calcium-enriched transcriptome was also seen in human dlPFC pyramidal cells from superficial layers, identified by the transcription factor CUX2. These experiments used single-cell transcriptomics from 50 neurotypical adults to identify and characterize subgroups of pyramidal cells that express high levels of CACNA1C (FIG. 3a). The CUX2-2 and CUX2-3 pyramidal cell subgroups were distinctive in expressing the highest levels of CACNA1C, in addition to co-expressing high levels of ADRB1 (β1-AR: FIG. 3B), CALB1 (calbindin: FIG. 3C) and KCNN3 (SK channel type 3: FIG. 3D), a potassium channel whose open state is modulated by calcium. These two subgroups also expressed high levels of GRIN2B (NMDAR-GluN2B: FIG. 3E), consistent with previous studies showing that macaque layer III dlPFC relies on NMDAR-GluN2B neurotransmission. The CUX2-2 and CUX2-3 subgroups also had moderate levels of HCN1 expression, although lower expression of KCNQ2, but had little expression of ADRB2 (β2-AR) compared to ADRB1 (FIG. 3B). The highly selective expression of CALB1 only in the CUX2-2 and CUX2-3 subgroups was especially noteworthy (FIG. 3C), as calbindin-expressing, layer III dlPFC pyramidal cells are the ones most vulnerable to tau pathology and degeneration in AD. Thus, these two subgroups of pyramidal cells with highly enriched calcium signaling may be especially vulnerable to neurodegeneration.

### Example 5: More Generalized Expression of Cav_{1.3}

The Cav_{1.3} LTCC isoform, encoded by CACNA1D, is also expressed in brain, and shares many properties with Cav_{1.2}, e.g. interacting with RyR on the SER to increase internal calcium release. Thus, the present study examined their distribution and expression in primate dlPFC as well. The transcriptomic data from human dlPFC pyramidal cells found that CACNA1D has much lower relative expression than CACNA1C for all dlPFC pyramidal cell subgroups. The immunoEM studies of macaque dlPFC found a more generalized expression pattern than for Cav_{1.2}. Thus, while Cav_{1.3} are expressed on layer III spines, dendrites and glia similar to Cav_{1.2}, they are also frequently expressed in axons. Axonal expression is consistent with seizures being a common symptom with genetic alterations in CACNA1D that increase Cav_{1.3} actions.

### Example 6: Functional Interactions in Rhesus dlPFC

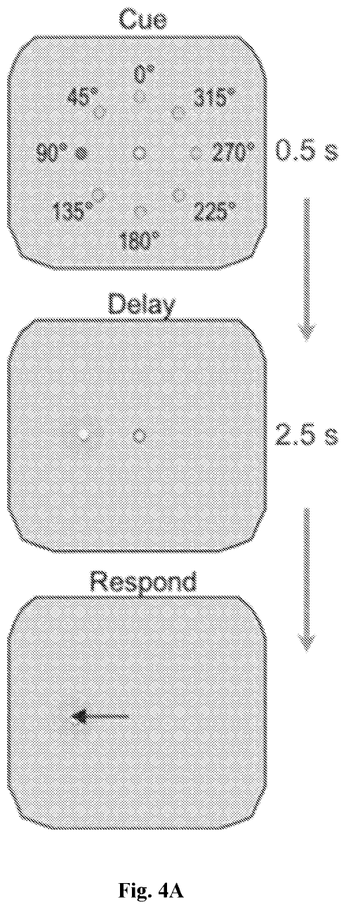

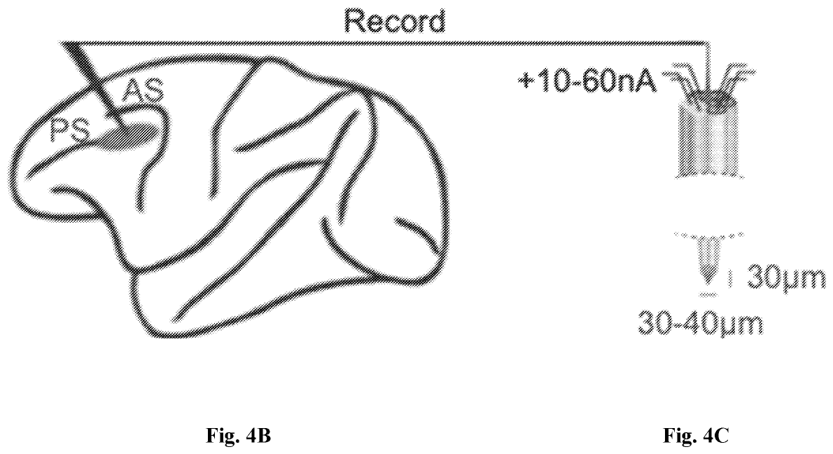

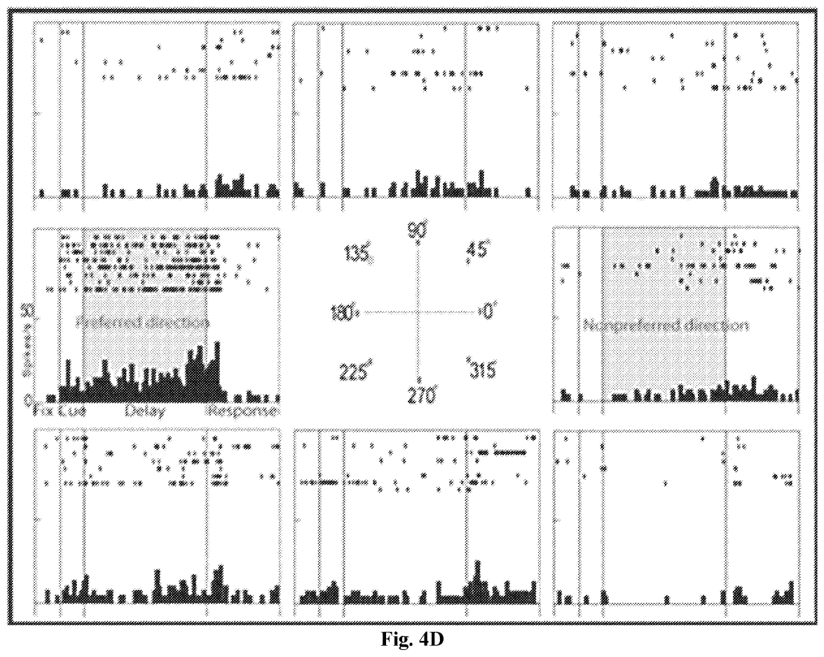

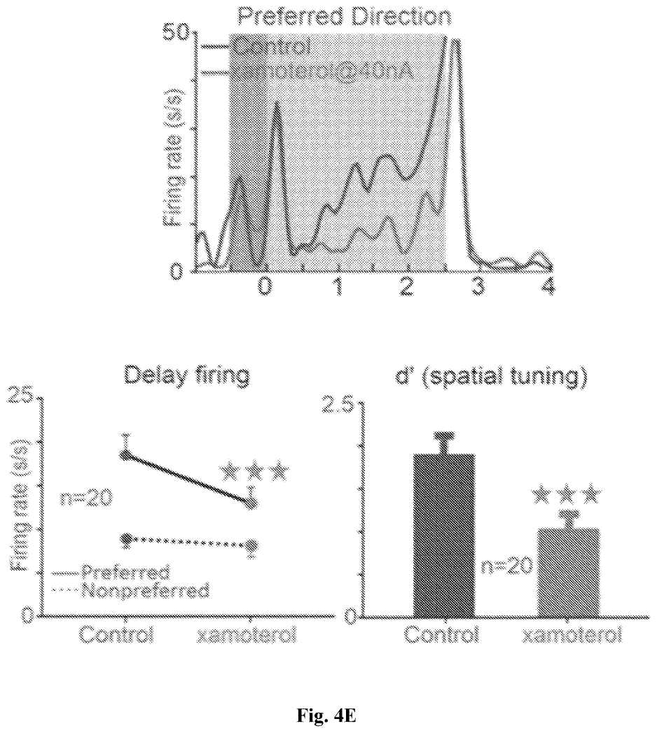

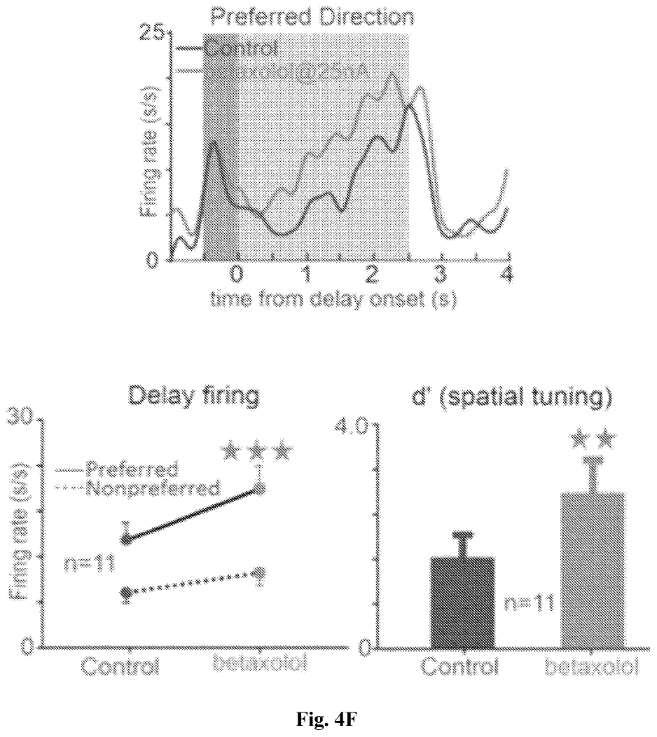

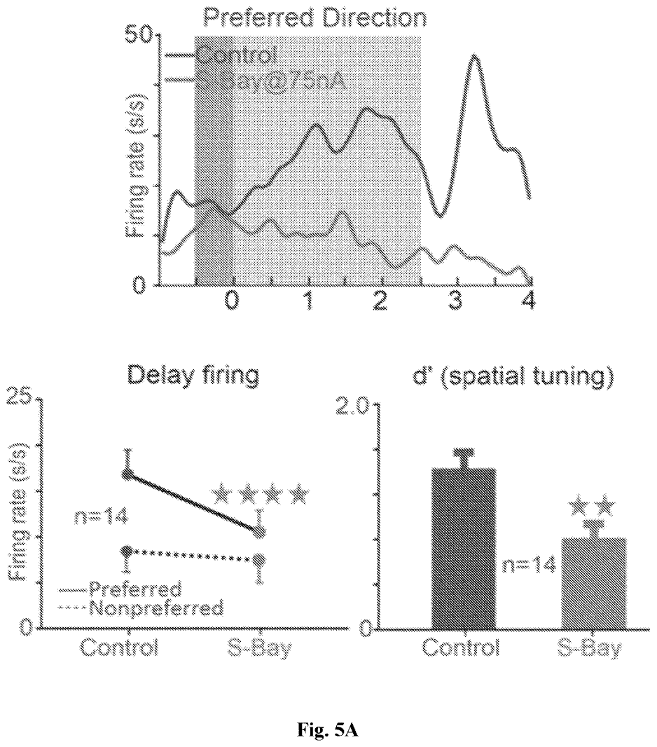

The physiological contributions of LTCC and β1-AR signaling were examined at the cellular level in rhesus macaques performing the oculomotor delayed response (ODR) spatial working memory task (FIG. 4A) to determine how they influence the neuronal firing needed for working memory and higher cognition. Recordings were made from the caudal principal sulcus (FIG. 4B) using an iontophoretic electrode (FIG. 4C) that allows minute ejection of charged compounds onto the neuron being recorded. The dlPFC contains “Delay cells” that are able to fire across the length of the delay period while the subject is remembering a location (FIG. 4D); this persistent firing is spatially tuned for the neuron's preferred direction, thus representing visual space in working memory. The effects of β1-AR-Cav_{1.2} signaling on dlPFC “Delay cell” firing were tested using single unit recordings coupled with iontophoresis in adult and aged rhesus monkeys performing the ODR task using repeated measures analyses. Aged monkeys have naturally-occurring calcium dysregulation, and thus are particularly useful for examining agents that reduce calcium signaling. As there are no compounds selective for Cav_{1.2}, these experiments used agents targeting all LTCCs (i.e. Cav_{1.2} and Cav_{1.3} in dlPFC).

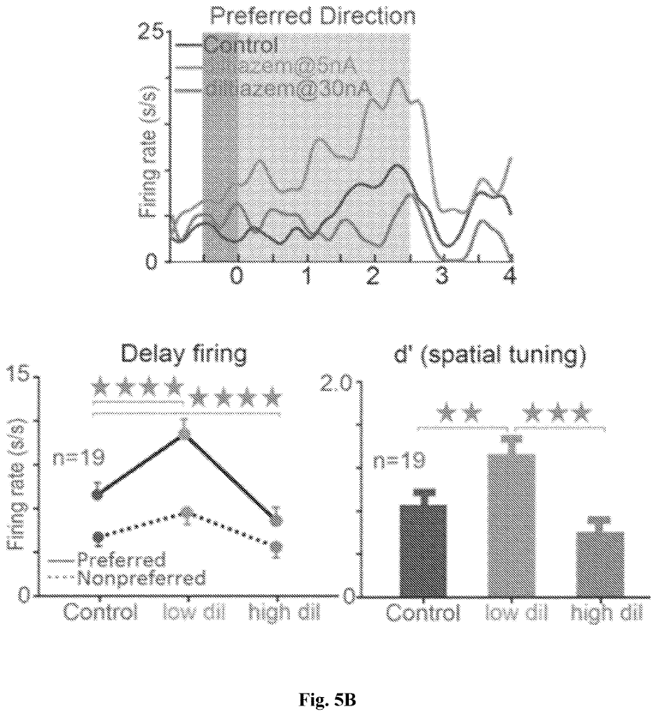

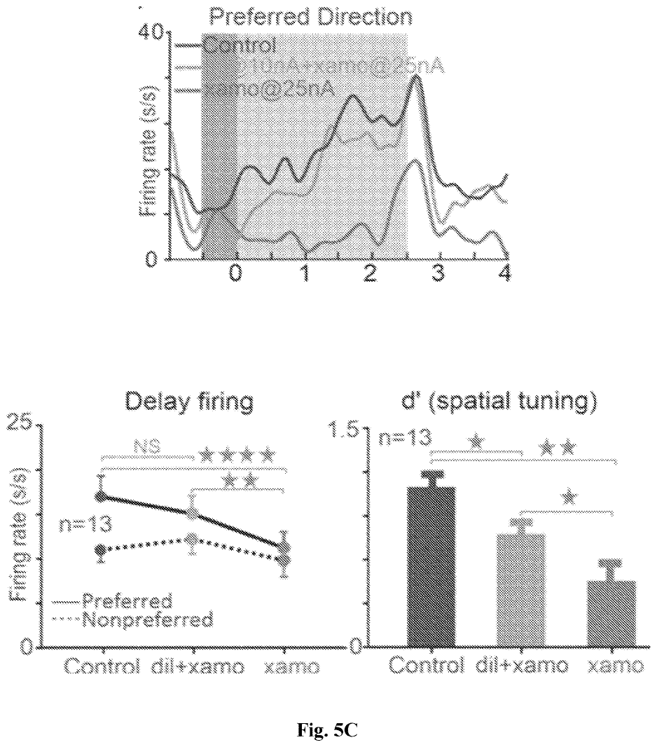

Iontophoresis of a β1-AR agonist (FIG. 4E) reduced Delay cell firing and d′ measures of spatial tuning (β1-AR agonist vs. control: n=20, reduction in delay firing for preferred direction p=0.003, reduction in d′ p=0.0002), while a β1-AR antagonist (FIG. 4F) increased delay-related firing and spatial tuning (β1-AR antagonist vs. control: n=11, increase in delay firing for preferred direction p=p=0.0009, increase in d′ p=0.0073). Similarly, iontophoresis of an LTCC agonist reduced delay firing and d′ measures of spatial tuning (FIG. 5A; LTCC agonist vs. control: n=19, reduction in delay firing for preferred direction p<0.0001, reduction in d′ p=0.0011), while a low dose of an LTCC antagonist enhanced Delay firing and spatial tuning (FIG. 5B, S8b: green: low dose LTCC antagonist vs. control: n=19, increase in delay firing for preferred direction p=0.0001, increase in d′ p=0.0016). The enhancing effects of the low dose LTCC antagonist were particularly evident across a wider dose range in the aged monkey, consistent with age-related calcium dysregulation contributing to loss of firing. These data from macaques resonate with the dlPFC working memory deficits seen in human subjects with genetic alterations in CACNA1C that could be normalized with LTCC blockade.

In contrast to the low dose enhancement, high doses of LTCC antagonist reduced neuronal firing and spatial tuning (FIG. 5B: high dose LTCC antagonist vs. control: n=19, reduction in delay firing for preferred direction p<0.0001, reduction in d′ p=0.0005). These data show that LTCCs are needed to maintain persistent firing, but that there is a very narrow, inverted-U for optimal dlPFC physiological function.

The physiological data also support a functional interaction between β1-AR and LTCCs similar to that in heart, where β1-AR initiation of PKA signaling opens Cav_{1.2} channels (FIG. 1B). As shown in FIG. 5C, the reduction in Delay cell firing induced by the β1-AR agonist, xamoterol, could be prevented (FIG. 5C) or reversed (S9b) by co-iontophoresis with the LTCC blocker, diltiazem (pretreatment blockade-LTCC antagonist+β1-AR agonist vs. β1-AR agonist alone: n=13. Delay firing: p=0.0015; β1-AR agonist vs. control, p<0.0001, d′: p=0.0025). Thus, LTCC opening contributes to the detrimental effects of β1-AR stimulation. As β1-AR have low affinity for NE, this detrimental mechanism may be mainly engaged under conditions with high NE release, e.g., under psychological or physiological stress.

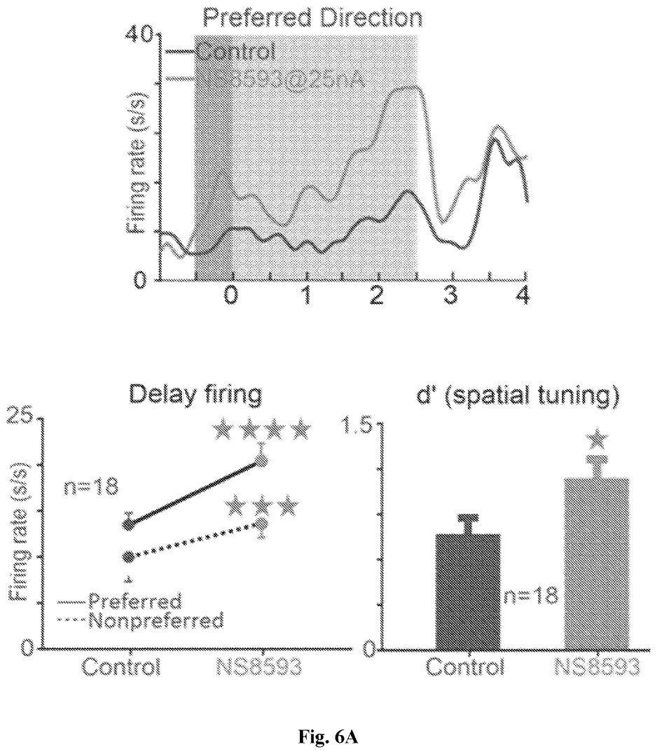

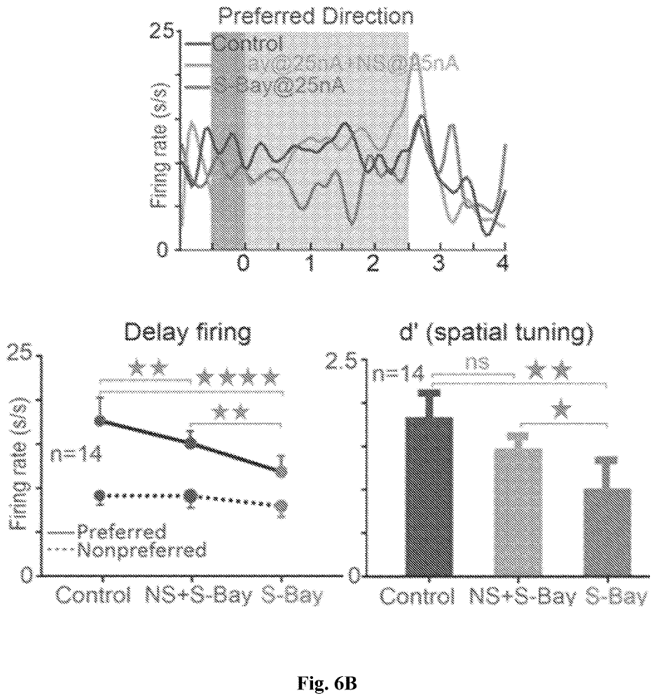

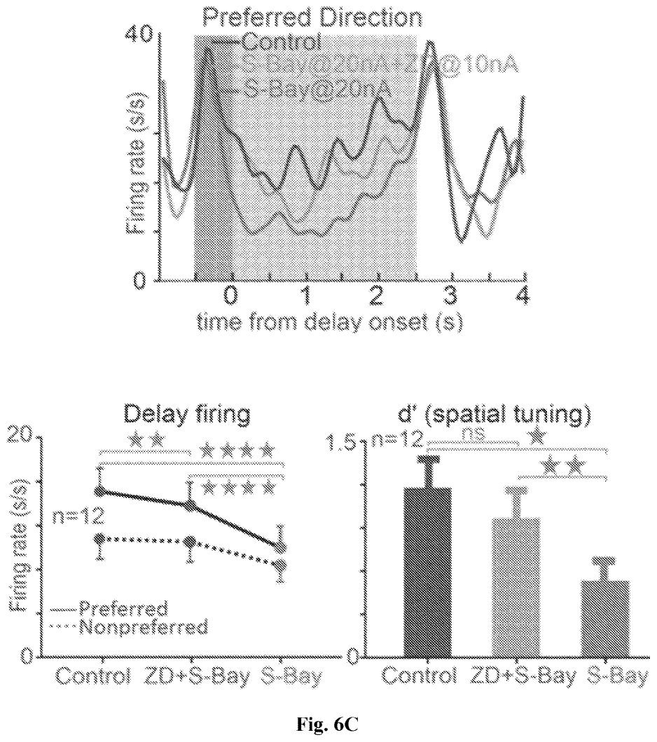

Further experiments examined the mechanisms by which increased LTCC opening reduces dlPFC Delay cell firing, testing the roles of SK and HCN channels known to reside on macaque layer III dlPFC spines (FIGS. 1G-1H), which are co-expressed in the dlPFC pyramidal cells in humans with the highest levels of CACNA1C (FIG. 2F). While it is common for HCN channels to reside on the distal dendrites of pyramidal cells, e.g., in V1, their expression on spines may be specialized to circuits that require dynamic changes in network connectivity, where they may open Slack K⁺ channels to rapidly weaken connectivity. It is already well-established that low dose HCN channel blockade enhances Delay cell firing. The current study provides the first evidence that low dose SK channel blockade with NS8593 also increases Delay cell firing and d′ measures of spatial tuning (FIGS. 6A: SK channel blockade vs. control: n=18, increase in delay firing for preferred direction p<0.0001, and a nonsignificant increase in firing for the nonpreferred directions, leading to an increase in d′ p=0.043).

The present study next tested whether blockade of SK or HCN channels would prevent the loss of firing produced by increasing the open state of LTCCs, as proposed in FIG. 1B. Consistent with this hypothesis, co-iontophoresis of the SK channel blocker, NS8593 (FIG. 6B), or the HCN channel blocker. ZD7288 (FIG. 6C), prevented the loss of Delay cell firing caused by the LTCC agonist, (S)-Bay-K6844 (LTCC agonist vs. SK channel blocker: n=14. LTCC agonist alone reduced firing compared to control p<0.0001, prevented by SK channel blockade p=0.0104, d′: p=0.0162; LTCC agonist vs. HCN channel blocker: n=12, LTCC agonist alone reduced firing compared to control p<0.0001, prevented by HCN channel blockade p<0.0001, d′: p=0.0051). Taken together, the physiological data show that LTCC opening is needed to support persistent neuronal firing without sensory stimulation, but that excessive levels driven by β1-AR stimulation reduce firing through the opening of SK and HCN channels. Sustained reductions in network connectivity and firing, e.g., due to alterations in CACNA1C that increase Cav_{1.2} expression, would likely spur synapse loss and cortical thinning, as has been seen in many CACNA1C-linked disorders.

### Example 7: Role in Stress-Induced Cognitive Deficits

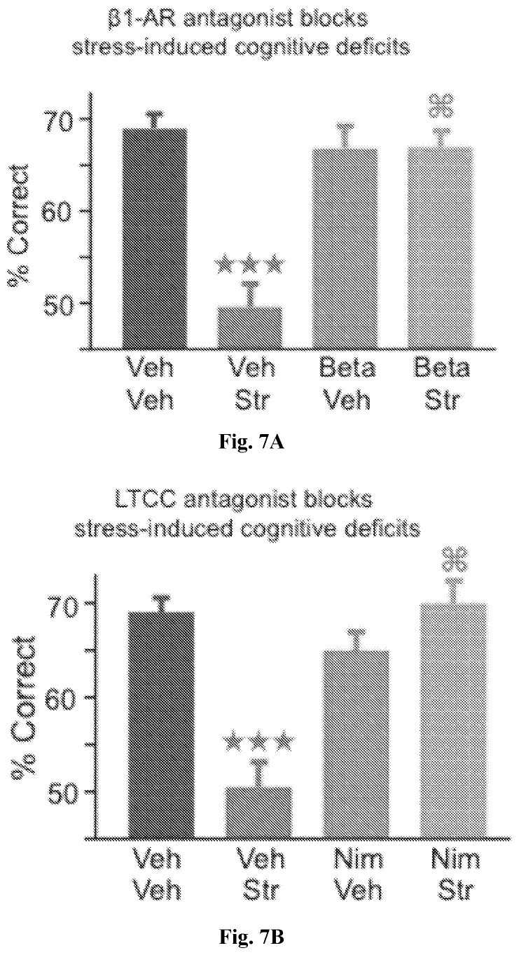

The final experiments examined whether systemic administration of compounds that block β1-AR or LTCCs prevent stress-induced working memory deficits in rhesus monkeys (n=6), as predicted by the physiological data. This experiment used the LTCC blocker, nimodipine, which normalizes dlPFC working memory in human subjects carrying the CACNA1C allele that increases channel expression. The current experiments tested the effects of β1-AR or LTCC blockade on stress-induced working memory deficits in adult monkeys performing the spatial delayed response task in a Wisconsin General Test Apparatus. The anxiogenic inverse benzodiazepine agonist, FG7142, was used as a pharmacological stressor, as it produces a classic stress response including cortisol release in humans and monkeys, yet allows precise dosing to produce a mild response that impairs accuracy without impeding motivation for testing for food rewards. Pretreatment with the β1-AR antagonist, betaxolol (FIG. 7A), or the LTCC blocker nimodipine (FIG. 7B), at doses that had no effect on their own, significantly blocked the deleterious effects of acute stress exposure (β1-AR antagonist vs. stress experiment-significant effect of stress: F(1,5)=187.5, p<0.0001: significant effect of betaxolol: F(1,5)=19.53, p=0.0069; significant stress×betaxolol interaction: F(1,5)=44.14, p=0.0012; post-hoc comparisons: vehicle+vehicle vs. vehicle+stress, p=0.001; vehicle+stress vs. betaxolol+stress, p=0.0008; LTCC vs. stress experiment-significant effect of stress: F(1,5)=15.86, p=0.011; significant effect of nimodipine: F(1,5)=16.4, p=0.0098; significant stress×nimodipine interaction: F(1,5)=67.24, p=0.0004; post-hoc comparisons: vehicle+vehicle vs. vehicle+stress, p=0.0009; vehicle+stress vs. nimodipine+stress, p=0.0006). These data support the hypothesis that β1-AR-Cav_{1.2} LTCC actions contribute to stress-induced cognitive dysfunction.

### Example 8

In one aspect, the current data identified a powerful mechanism for weakening dlPFC working memory function, where β1-AR drive on LTCCs reduced the firing of dlPFC Delay cells by increasing the open state of SK and HCN channels, all of which were localized on layer III dlPFC dendritic spines. While LTCCs were needed to sustain the delay-related firing, similar to the need for NMDAR-GluN2B neurotransmission, higher levels of LTCC activation markedly reduced neuronal firing. Importantly, Cav_{1.2} and β1-AR were co-localized in calbindin-expressing layer III pyramidal cells, which are known to be particularly vulnerable to tau pathology when they lose calbindin with advancing age. A parallel pattern of expression was found in human dlPFC, where the subgroups of pyramidal cells from superficial layers that expressed the highest levels of CACNA1C (Cav_{1.2}), also expressed ADRB1 (β1-AR), KCNN3 (SK3 channels), GRIN2B (NMDAR-GluN2B) and CALB1 (calbindin), with CACNA1C the predominant LTCC. As either inadequate or excessive LTCC actions reduced the dlPFC neuronal firing needed for working memory, these data help to explain why either loss- or gain-of-function mutations in CACNA1C would impair dlPFC activation and connectivity in human subjects, and increase risk of mental disorders linked to deficits in dlPFC cognition and top-down control of emotion.

Pyramidal Cell Heterogeneity and Roles in dlPFC Cellular Physiology

The current study showed that moderate levels of LTCC opening are essential to dlPFC persistent firing, as high dose blockade markedly reduced Delay firing. These data are resonant with genetic studies in rodents, where heterozygous deletion of CACNA1C impaired PFC reversal learning. An enrichment in calcium-related signaling in layer III dlPFC may reflect the need for greater calcium concentrations to maintain depolarization of the PSD to support NMDAR-mediated persistent firing, including NMDAR with GluN2B subunits (GRIN2B) that flux high levels of calcium, and are localized within the macaque PSD. The current study found that Cav_{1.2} are also near or within the PSD, often near the SER spine apparatus, and thus may help to depolarize the PSD during delay-related firing. Interestingly, CACNA1C is enriched in macaque layer III dlPFC (callosal-projecting) and parietal (ipsilateral-projecting) circuits interconnected with dlPFC, suggesting that heightened calcium may be especially needed to sustain recurrent excitation in long-range recurrent excitatory circuits.

The transcriptomic analysis of the human dlPFC was consonant with data from macaques, and revealed a surprising degree of heterogeneity between dlPFC pyramidal cells. It was particularly striking that there were only two subgroups of pyramidal cells that expressed CALB1 (calbindin), and that these same subgroups expressed a calcium-enriched transcriptome including the highest levels of CACNA1C, as well as GRIN2B, encoding the GluN2B subunit of the NMDAR that fluxes the highest levels of calcium, and KCNN3, encoding the SK3 potassium channel whose open state is increased by calcium. The CUX2-2 subgroup in particular, expressed very high levels of GRIN2B, KCNN3 and ADRB1, a combination which the macaque physiology suggests would make these cells particularly vulnerable to stress.

The macaque physiological data, coupled with the immunoEM, indicate that the stress response in primate dlPFC shares striking homologies to the stress response in heart. β1-AR have low affinity for NE, and thus are usually engaged under conditions of high NE release, e.g. during stress. In heart. β1-AR activate Cav_{1.2} currents, which drive calcium release from the sarcoplasmic reticulum to increase muscle contractility. The current study showed evidence for a parallel mechanism in primate layer III dlPFC, where β1-AR, Cav_{1.2} and SK3 channels are all localized on spines near the SER, and β1-AR stimulation in macaque dlPFC reduced neuronal firing by increasing LTCC actions, and the open state of SK and HCN channels. This mechanism would allow rapid disconnection of the recurrent excitatory connections needed to sustain neuronal firing during working memory, which may have survival value under some dangerous conditions.

### Relevance to Stress-Related Cognitive Dysfunction

dlPFC function is remarkably vulnerable to stress exposure, where even a mild, uncontrollable stress rapidly impairs dlPFC function in humans and monkeys, while strengthening the amygdala and the salience network, reconfiguring the brain into a less thoughtful, more reactive state. Furthermore, dlPFC circuits atrophy or degenerate in multiple mental disorders where stress is an etiological risk, including depression and PTSD, substance abuse, schizophrenia, and Alzheimer's disease (AD). Thus, understanding the molecular mechanisms that cause stress-induced dlPFC deficits has both societal and clinical implications. The current study performed behavioral studies in macaques that confirmed a role for β1-AR-LTCC signaling in stress-induced cognitive deficits, as systemic administration of either a β1-AR or LTCC antagonist blocked the working memory deficits caused by a pharmacological stressor. These data contrast with previous research in humans and animals showing that β1-AR stimulation enhances the activity of the amygdala and the salience network during stress. The current data are the first to show that β1-AR have an opposite effect in dlPFC, reducing dlPFC neuronal firing and working memory function, indicating a potential therapeutic target. As the strength of dlPFC connectivity is related to the ability to control emotions during stress, loss of dlPFC function with increased β1-AR-LTCC signaling would be particularly deleterious to thoughtful, top-down regulation of behavior. This may be particularly problematic with chronic stress and/or inflammation, when Cav_{1.2} expression is increased, e.g. in the PFC. Taken together, these data may help to explain the rise in stress-related disorders during the COVID19 pandemic, such as depression, substance abuse, and violence, that are worsened by weaker PFC top-down control.

### Relevance of LTCCs to Human Cognition and Cognitive Disorders

In contrast to the constellation of symptoms linked to alterations in CACNA1D, genetic alterations in CACNA1C are especially linked to impaired dlPFC activation, connectivity and function. They are also associated with increased risk of dlPFC-associated mental disorders such as schizophrenia, depression, bipolar disorder, ADHD and PTSD. Given the current findings that β1-AR/LTCC signaling contributes to stress-induced working memory deficits, it is noteworthy that psychological and physiological stressors are risk factors, or actually causal, for all these disorders. It is also noteworthy that dendritic spine loss in schizophrenia is specific to layer III, and this is the layer where pyramidal cells that co-express Cav_{1.2} and calbindin are concentrated in macaque dlPFC.

Finally, the current data are particularly relevant to the etiology of sporadic AD, as they identify the molecular signatures of the subgroups of dlPFC pyramidal cells most vulnerable to AD neurodegeneration. Calbindin-containing pyramidal cells in dlPFC layer III are selectively vulnerable to ensuing tau pathology and neurodegeneration, when calbindin is lost with age. The current human transcriptional data demonstrate very selective expression of CALB1 (calbindin) in just two subgroups of pyramidal cells-CUX2-2 and CUX2-3-that express a calcium-enriched profile with the highest levels of CACNA1C. This pattern is consonant with both genomic data identifying an interaction of CACNA1C and the calcium channel ryanodine receptor RYR3 as a risk factor for AD, and a variety of data showing that calcium dysregulation increases AD pathology and degeneration, including evidence that ApoE4 (but not ApoE3) increases sustained calcium entry through LTCCs. The current data also help to explain why psychological and physiological stressors are risk factors for sporadic AD, as they would drive LTCC calcium signaling. Thus, the magnification of calcium signaling needed for higher cognition in recently evolved dlPFC pyramidal cell circuits would become toxic when regulation is lost with stress and age.

### Example 9: Anti-Stress Effects Displayed by Betaxolol are Universal to β1-AR Antagonists

To exclude the unlikely possibility that the anti-stress effect of the β1-AR antagonist betaxolol was not universal to β1-AR antagonists but rather specific to betaxolol, another β1-AR antagonist, nebivolol, was tested, as well.

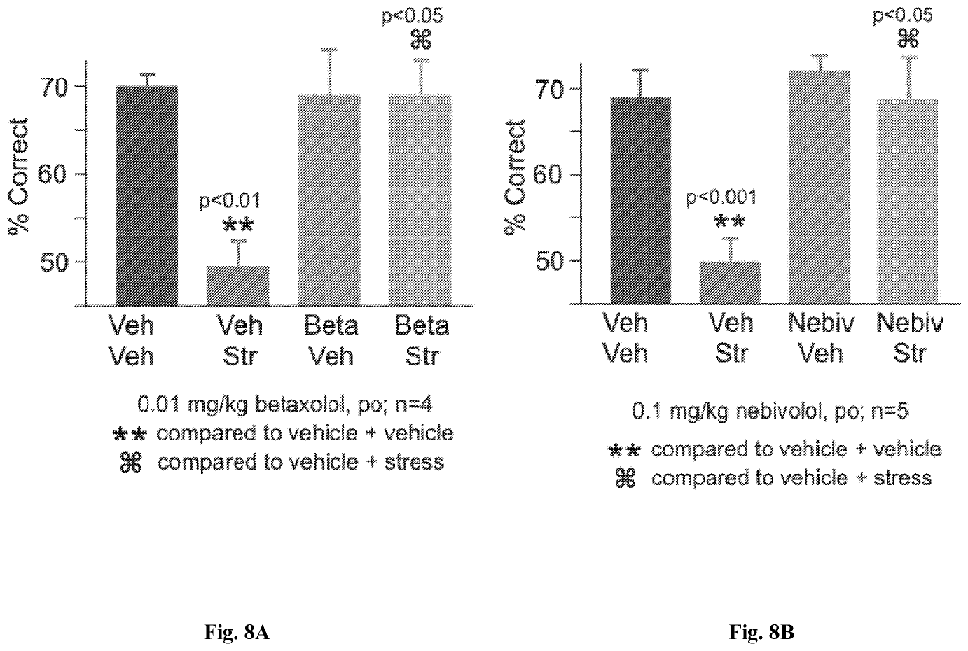

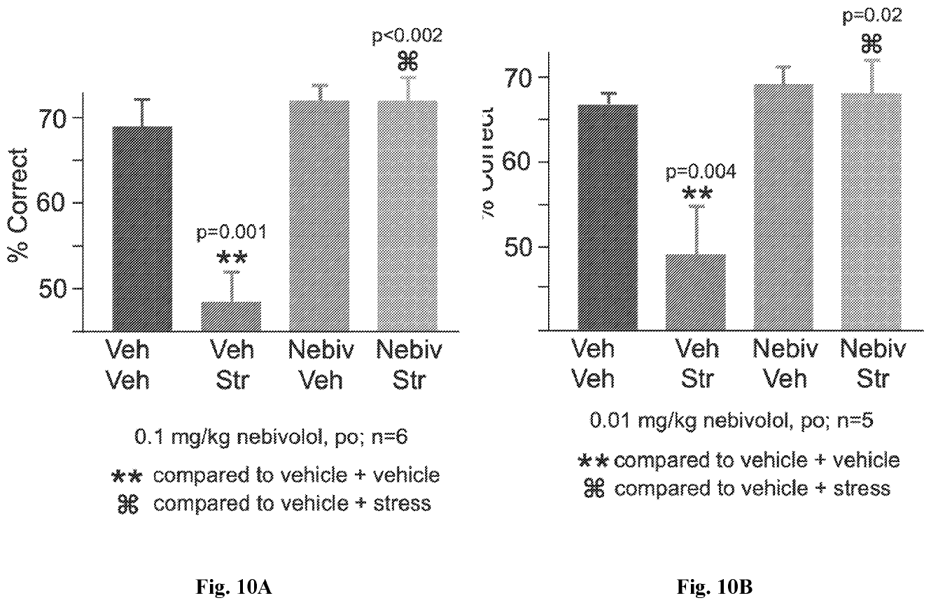

Referring to FIGS. 8A-8B, the anti-stress effects of the β1-AR antagonist betaxolol described in FIG. 7A is not limited to betaxolol only, but universal to specific inhibitions of β1-AR, in accordance with some embodiments. FIG. 8A: betaxolol (β1-AR specific antagonist used in FIG. 7A) prevented working memory deficits in FG7142-treated animals. FIG. 8B: another β1-AR specific antagonist, nebivolol, also prevented working memory deficits in FG7142-treated animals. Referring to FIGS. 10A-10B, additional nebivolol tests were performed on more animals. The results confirm that the anti-stress effects of β1-AR antagonists in general.

### Example 10:31-AR Selectivity is Desirable for Anti-Stress Effects of β-AR Antagonists

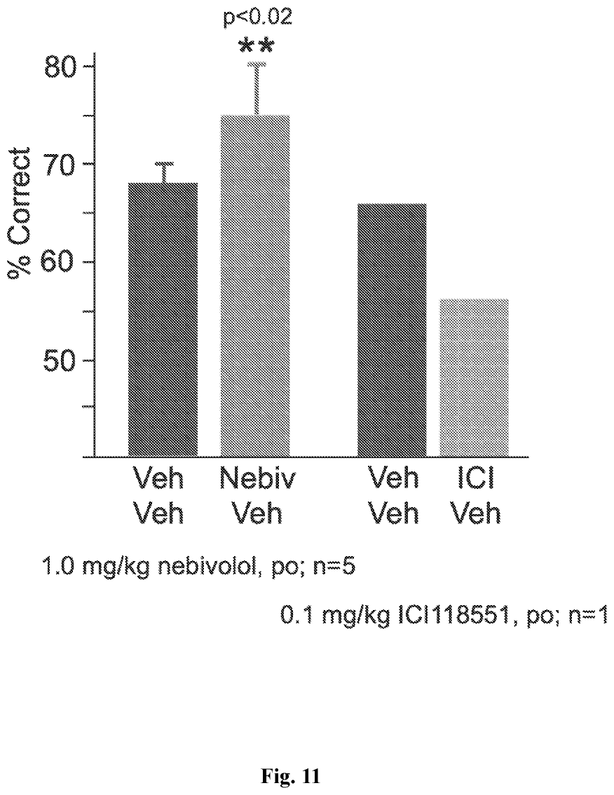

Referring to FIG. 11, the β1-AR antagonist nebivolol again displayed anti-stress effects. The β2-AR antagonist, IC1118551, however; appeared to have impaired working memory in the test animals (n=1). This results underscore the impotence of β1-AR selectivity, as the inhibition of β2-AR appeared to caused the opposite effects.

### Example 11

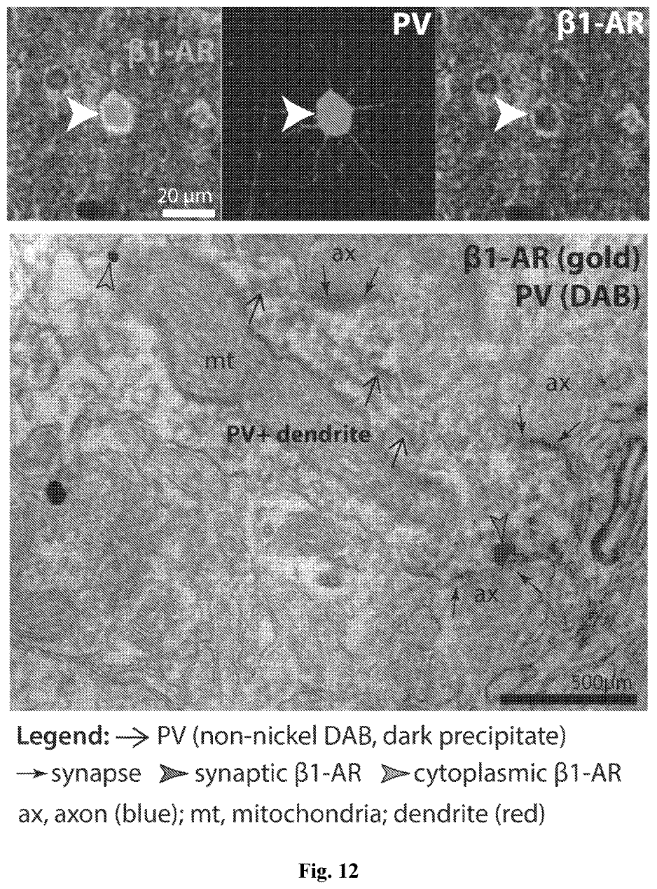

Referring to FIG. 12, the anatomy figure shows that β1-AR is located on parvalbumin (PV) expressing GABAergic interneurons in the primate dorsolateral prefrontal cortex. Without wishing to be bound by theory, it is hypothesized that the β1-AR there may drive the inhibition of prefrontal cognitive function.

### Enumerated Embodiments

In some aspects, the present disclosure is directed to the following non-limiting embodiments:
Embodiment 1: A method of treating, ameliorating, and/or preventing a stress-related disorder in a subject in need thereof, the method comprising administering to the subject a therapeutically effective amount of a selective β-adrenergic receptor antagonist compound, wherein the compound has a larger dissociation constant (pK_{D}) for the β1-adrenergic receptor subtype (β1-AR) than for the β2-adrenergic receptor subtype (β2-AR) and/or β3-adrenergic receptor subtype (β3-AR).
Embodiment 2: A method of treating, ameliorating, and/or preventing an anxiety disorder in a subject in need thereof, the method comprising administering to the subject a therapeutically effective amount of a selective β-adrenergic receptor antagonist compound, wherein the compound has a larger dissociation constant (pK_{D}) for the β1-adrenergic receptor subtype (β1-AR) than for the β2-adrenergic receptor subtype (β2-AR) and/or β3-adrenergic receptor subtype (β3-AR).
Embodiment 3: The method of any one of Embodiments 1-2, wherein the compound has a larger pK_{D} for the β1-adrenergic receptor subtype (β1-AR) than for the β2-adrenergic receptor subtype (β2-AR).
Embodiment 4: The method of any one of Embodiments 1-3, wherein the compound has an inhibition constant (pK_{D}) for the β1-adrenergic receptor subtype (β1-AR) that is 0.3 units or higher than for the β2-adrenergic receptor subtype (β2-AR) and/or β3-adrenergic receptor subtype (β3-AR).
Embodiment 5: The method of any one of Embodiments 1-4, wherein the selective β1-adrenergic receptor subtype antagonist compound comprises at least one selected from the group consisting of acebutolol, atenolol, betaxolol, bisoprolol, CGP 20712A, esmolol, ICI 89406, metoprolol, nebivolol, and practolol.
Embodiment 6: The method of any one of Embodiments 1 and 3-5, wherein the stress-related disorder comprises a stress-induced prefrontal cortical dysfunction.
Embodiment 7: The method of any one of Embodiments 1 and 3-6, wherein the stress-related disorder is at least one selected from the group consisting of post-traumatic stress disorder (PTSD), reactive attachment disorder, disinhibited social engagement disorder, acute stress disorder, adjustment disorder, and an agitation/disruptive/aggressive behavior associated with dementia.
Embodiment 8: The method of any one of Embodiments 2-5, wherein the anxiety disorder is at least one selected from the group consisting of generalized anxiety disorder (GAD), panic disorder, phobia, agoraphobia, separation anxiety disorder, and social anxiety disorder.
Embodiment 9: The method of any one of Embodiments 1-8, wherein the compound is administered by at least one route selected from the group consisting of oral, nasal, rectal, intravaginal, parenteral, buccal, sublingual, and topical.
Embodiment 10: The method of any one of Embodiments 1-9, wherein the selective β-adrenergic receptor antagonist compound is the only compound administered to the subject to treat, ameliorate, and/or prevent the stress-related and/or anxiety disorder.
Embodiment 11: The method of any one of Embodiments 1-10, wherein the subject is a mammal.
Embodiment 12: The method of any one of Embodiments 1-11, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating, or preventing a stress-related disorder or an anxiety disorder in a subject in need thereof, the method comprising administering to the subject a therapeutically effective amount of a selective β-adrenergic receptor antagonist compound, wherein the compound has a larger dissociation constant (pK_{D}) for the β1-adrenergic receptor subtype (β1-AR) than for the β2-adrenergic receptor subtype (β2-AR) or β3-adrenergic receptor subtype (β3-AR).

2. (canceled)

3. The method of claim 1, wherein the compound has a larger pK_{D} for the β1-adrenergic receptor subtype (β1-AR) than for the β2-adrenergic receptor subtype (β2-AR).

4. The method of claim 1, wherein the compound has an inhibition constant (pK_{D}) for the β1-adrenergic receptor subtype (β1-AR) that is 0.3 units or higher than for the β2-adrenergic receptor subtype (β2-AR) or β3-adrenergic receptor subtype (β3-AR).

5. The method of claim 1, wherein the selective β1-adrenergic receptor subtype antagonist compound comprises at least one selected from the group consisting of acebutolol, atenolol, betaxolol, bisoprolol, CGP 20712A, esmolol, ICI 89406, metoprolol, nebivolol, and practolol.

6. The method of claim 1, wherein the stress-related disorder comprises a stress-induced prefrontal cortical dysfunction.

7. The method of claim 1, wherein the stress-related disorder is at least one selected from the group consisting of post-traumatic stress disorder (PTSD), reactive attachment disorder, disinhibited social engagement disorder, acute stress disorder, adjustment disorder, and an agitation/disruptive/aggressive behavior associated with dementia.

8. The method of claim 1, wherein the anxiety disorder is at least one selected from the group consisting of generalized anxiety disorder (GAD), panic disorder, phobia, agoraphobia, separation anxiety disorder, and social anxiety disorder.

9. The method of claim 1, wherein the compound is administered by at least one route selected from the group consisting of oral, nasal, rectal, intravaginal, parenteral, buccal, sublingual, and topical.

10. The method of claim 1, wherein the selective β-adrenergic receptor antagonist compound is the only compound administered to the subject to treat, ameliorate, or prevent the stress-related or anxiety disorder.

11. The method of claim 1, wherein the subject is a mammal.

12. The method of claim 1, wherein the subject is a human.
